(19) United States
(12) Patent Application Publication
(10) Pub. No.: US 2019/0240130 A1
(43) Pub. Date: Aug. 8, 2019

(54) HAIR COSMETIC COMPOSITION COMPRISING THIOL-BASED COMPOUNDS

(71) Applicant: L'CREAL, Paris (FR)

(72) Inventors: Marina Tavares GREGOLIN, Rio de Janeiro (BR); Ana Paula Leme DE MAGALHAES, Rio de Janeiro (BR); Bruno Maiko SATO, Rio de Janeiro (BR); Erika Alegrio Jarque PETALI, Rio de Janeiro (BR); Sintia Aguiar MARTINS, Rio de Janeiro (BR); Diego ALVES, Rio de Janeiro (BR)

(21) Appl. No.: 16/338,118

(22) PCT Filed: Sep. 30, 2016
(86) PCT No.: PCT/BR2016/050245

§ 371 Date: Mar. 29, 2019

(51) Int. Cl. A61K 8/41; A61K 8/46; A61K 8/34; A61K 8/898; A61Q 5/12

(52) U.S. Cl. CPC A61K 8/416; A61K 8/46; A61K 8/345; A61K 8/92; A61K 8/342; A61Q 5/12; A61K 2800/5426; A61K 8/898

(57) ABSTRACT

The present invention relates to compositions for conditioning hair fibers, in particular human hair fibers, comprising (a) cationic surfactants; (b) thiol-based compounds; (c) fatty alcohols in an amount of from about 6% to about 20% by weight; (d) a component selected from polyols, fatty substances other than a fatty alcohol, and mixtures thereof; (e) water; and (f) optionally, neutralizing agents, wherein the pH of the composition ranges from 2 to less than 7. The invention also concerns a process for imparting hair care benefits to hair fibers such as straightening effects, volume reduction, fizz control, manageability, cosmeticity, and smooth feel, in addition to conditioning benefits and a use for hair care employing the cosmetic compositions of the present invention.

### FIELD OF THE INVENTION

The present application relates to cosmetic compositions for use on keratinous substrates, such as keratin fibers. In particular, it relates to compositions and methods for conditioning hair and providing cosmetic and caring benefits to hair.

### BACKGROUND OF THE INVENTION

The quality and condition of hair is generally adversely affected by the action of external agents such as sunlight, wind, pollution, humidity, and chemicals in swimming pools, and also by mechanical or chemical treatments, such as brushing, combing, shampooing, dyeing, bleaching, permanent-waving and/or relaxing. Hair is thus damaged by these various factors and may over time become dry, coarse or dull, especially in fragile areas, and more particularly at the ends leading to split ends. Under such circumstances, hair can also become less manageable, more frizzy and less disciplined, and more difficult to style or shape.

Hair care products are used to combat these drawbacks. Conventional cleansing compositions such as shampoos, for example, which contain surfactants such as anionic, nonionic and/or amphoteric type surfactants, can be employed to remove the diverse types of soils typically present on hair.

These cleansing compositions, while providing good cleansing power, may result in a less conditioned or rough feel to the hair due to, for example, the gradual removal of the natural or applied fats, lipids, or proteins contained in or at the surface of the hair. In addition, washing hair with water alone can also cause the hair to become tangled, less smooth and dry.

Thus, a second composition, a hair conditioner or hair masque (mask) product, can be used on hair after it has been shampooed or rinsed in order to confer hair caring properties to hair. Traditional conditioners and masques provide conditioning, smoothing, softening and visual sleekness to hair and are generally rinse-off products. However, consumers continuously seek better and unique hair caring regimens and products such as conditioners that can control, or even eliminate frizziness, and also control or reduce the volume and the apparent mass of hair on the head. Consumers also desire such regimens and products to be able to provide additional benefits of changing the appearance, shape or configuration of hair.

One type of product that can shape or configure hair are hair styling products, for example, leave-in gels and creams which employ ingredients such as film forming polymers that impart desirable styling or shaping hold while on the hair. However, some ingredients can be easily removed from the hair, for example by rinsing or washing. Thus, any cosmetic benefits to the hair from such products are generally diminished or removed once the hair is rinsed or washed.

Another type of composition that can be applied onto hair in order to change its shape and make it more manageable is an alkaline composition that is found in commercial hair relaxers or hair straighteners. These products can confer longer lasting shape or style to the hair. Alkaline hair relaxing/straightening involves hydrolyzing the keratin of the hair with various alkaline agents, such as inorganic hydroxides, for instance sodium hydroxide, or organic hydroxides, such as guanidine hydroxide, or organic amines. Hair relaxing/straightening products that employ sodium hydroxide or potassium hydroxide are also called lye-based products and products that use other alkaline agents such as lithium hydroxide, calcium hydroxide, organic hydroxides and other non-hydroxide compounds, for example, organic amines, generally fall under the category of no-lye products. Still, it is desirable to find alternatives to the alkaline lye- and no-lye-based products and process described above which can damage the hair by weakening and/or causing dryness of the hair fibers.

Thus, it is highly desirable to have hair care regimens and products that, for example, relax or straighten curly or kinky hair, including wavy hair, or reduce/loosen curls while making the hair more manageable, untangled, more easily styled or shaped, and disciplined. At the same time, it is also highly desirable that such regimens and products confer cosmetic effects that are long-lasting, that is, the effects remain even after repeated washings or cleansing.

It is an object of embodiments of the disclosure to provide a rinse-off hair cosmetic composition with ingredients that can effectively condition the hair and at the same time, impart good styling/shaping or straightening benefits as well as enhanced hair caring and manageability properties and other cosmetic properties such as volume control/reduction, frizz control/reduction, discipline (less fly-aways), and regularity of shape.

However, the discovery of new compositions and processes for treating hair that have enhanced efficacy but impart less or minimal damage to hair, may pose challenges to manufacturers and formulators because the incorporation of new ingredients into the compositions may negatively impacting their performance, cosmetic attributes, texture, and formulation stability. In addition, the acidity/alkalinity and/or pH is an important consideration for these products. New processes for treating and changing the shape of hair may also impact the performance of the compositions, processing times and quality of use. Thus, manufacturers of such products continuously test the use of new raw materials and ingredients or new product forms and seek to re-formulate and create new products with the desired qualities, while still remaining stable and safe to use.

### SUMMARY OF THE INVENTION

The present invention is directed to a hair cosmetic agent composition, the composition comprising:

(a) at least one cationic surfactant;

(b) at least one thiol-based compound selected from thiolactic acid, thiolactic acid derivatives, their salts, and mixtures thereof;

(c) at least one fatty alcohol in an amount of from about 6% to about 20% by weight;

(d) at least component selected from (i) polyols, (ii) fatty substances other than a fatty alcohol, and mixtures thereof in an amount of from about 0.1% to about 10% by weight;

(e) water; and

(f) optionally, at least one neutralizing agent;

wherein the pH of the composition ranges from about 2 to less than 7;

all weights being relative to the total weight of the composition.

The present invention is also directed to processes for treating the hair involving the application of the above-described composition onto hair, and including a step of heating the treated hair at a temperature of equal to or greater than 50° C.

### BRIEF DESCRIPTION OF THE DRAWINGS

Implementations of the present technology will now be described, by way of example only, with reference to the attached figures, wherein:

FIG. 1 provides images of hair swatches treated according to one application or treatment cycle including applying inventive or comparative conditioner formulas onto hair and heating the hair.

FIG. 2 provides images of hair swatches treated according to two application or treatment cycles including applying inventive or comparative conditioner formulas onto hair and heating the hair.

FIG. 3 provides images of hair swatches treated according to five application or treatment cycles including applying inventive or comparative conditioner formulas onto hair and heating the hair.

FIG. 4 provides images of hair swatches treated according to one application or treatment cycle including applying inventive or comparative silicone containing conditioner formulas onto hair and heating the hair.

FIG. 5 provides images of hair swatches treated according to one application or treatment cycle including applying inventive or comparative masque formulas onto hair and heating the hair.

FIG. 6 provides images of hair swatches treated according to two application or treatment cycles including applying inventive or comparative masque formulas onto hair and heating the hair.

FIG. 7 provides images of hair swatches treated according to five application or treatment cycles including applying inventive or comparative masque formulas onto hair and heating the hair.

It should be understood that the various aspects are not limited to the arrangements and instrumentality shown in the drawings.

### DETAILED DESCRIPTION OF THE INVENTION

The present compositions effectively condition the hair and provide manageability properties to hair, including one or more of straightening effects; frizz control; volume reduction or volume control; styling or shaping effects; curling effects; texlaxing effects or reduction/loosening of curls; improvement or retention of curl definition; humidity resistance; cosmeticity to the feel; smooth feel; natural feel; less or reduced rough ends; and/or improvement of the appearance of hair.

“At least one” as used herein means one or more and thus includes individual components as well as mixtures/combinations.

“Keratinous substrates” as used herein, includes, but is not limited to keratin fibers such as hair and/or scalp on the human head.

“Conditioning” as used herein means imparting to one or more hair fibers at least one property chosen from combability, moisture-retentivity, luster, shine, and softness. The state of conditioning can be evaluated by any means known in the art, such as, for example, measuring, and comparing, the ease of combability of the treated hair and of the untreated hair in terms of combing work (gm-in), and consumer perception.

The term “treat” (and its grammatical variations) as used herein refers to the application of the compositions of the present disclosure onto the surface of keratinous substrates such as hair. The term “treat” (and its grammatical variations) as used herein also refers to contacting keratinous substrates such as hair with the compositions of the present disclosure.

A “rinse-off” product refers to a composition such as a hair care composition that is rinsed and/or washed with water either after or during the application of the composition onto the keratinous substrate, and before drying and/or styling said keratinous substrate. At least a portion of the composition is removed from the keratinous substrate during the rinsing and/or washing.

A “leave-on” (also called leave-in) product refers to a cosmetic composition such as a hair cosmetic composition that is applied to a keratinous substrate such as hair and not further subjected to a rinsing and/or washing step for at least 1 hour or for a period of time ranging from 1 hour up to 72 hours such as from 4 hours up to 48 hours or from 8 hours up to 36 hours or from 8 hours up to 24 hours.

The term “stable” as used herein means that the composition does not exhibit phase separation and/or crystallization.

“Volatile”, as used herein, means having a flash point of less than about 100° C.

“Non-volatile”, as used herein, means having a flash point of greater than about 100° C.

“Reducing agent” as used herein, means an agent capable of reducing the disulfide bonds of the hair.

“Active material” as used herein with respect to the percent amount of an ingredient or raw material, refers to 100% activity of the ingredient or raw material.

“Substituted,” as used herein, means comprising one or more substituents. Non-limiting examples of substituents include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Polymers,” as defined herein, include homopolymers and copolymers formed from at least two different types of monomers.

“INCI” is an abbreviation of International Nomenclature of Cosmetic Ingredients, which is a system of names provided by the International Nomenclature Committee of the Personal Care Products Council to describe personal care ingredients.

The compositions and methods of the present invention can comprise, consist of, or consist essentially of the essential elements and limitations of the invention described herein, as well as any additional or optional ingredients, components, or limitations described herein or otherwise useful.

Other than in the operating examples, or where otherwise indicated, all numbers expressing quantities of ingredients and/or reaction conditions are to be understood as being modified in all instances by the term “about,” meaning within+/−5% of the indicated number.

All percentages, parts and ratios herein are based upon the total weight of the compositions of the present invention, unless otherwise indicated.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

In an embodiment, the present invention is directed to a hair cosmetic agent composition, the composition comprising:

(a) at least one cationic surfactant;

(b) at least one thiol-based compound selected from thiolactic acid, thiolactic acid derivatives, their salts, and mixtures thereof;

(c) at least one fatty alcohol in an amount of from about 6% to about 20% by weight;

(d) at least component selected from (i) polyols, (ii) fatty substances other than a fatty alcohol, and mixtures thereof in an amount of from about 0.1% to about 10% by weight;

(e) water; and

(f) optionally, at least one neutralizing agent;

wherein the pH of the composition ranges from about 2 to less than 7;

all weights being relative to the total weight of the composition.

In one embodiment of the present invention, the at least one cationic surfactant is selected from optionally polyoxyalkenylated primary, secondary, tertiary fatty amine salts, quaternary ammonium compounds and their salts, and mixtures thereof, and preferably from quaternary ammonium compounds and their salts, and more preferably from cetrimonium chloride, behentrimonium chloride, dipalmitoylethylhydroxyethylmonium methosulfate, and mixtures thereof.

In one embodiment of the present invention, the at least one thiol-based compound is selected from thiolactic acid.

In one embodiment of the present invention, the at least one thiol-based compound is present in an amount of from about 0.5% to about 15% by weight, preferably from about 0.6% to about 14% by weight, more preferably from about 0.7% to about 12% by weight, even more preferably from about 0.8% to about 10% by weight or from about 1% to about 8% by weight, based on the total weight of the composition.

In one embodiment of the present invention, the at least one fatty alcohol is selected from linear saturated/unsaturated fatty alcohols comprising from 6 to 60 carbon atoms and preferably from cetyl alcohol, stearyl alcohol, cetearyl alcohol (mixture of cetyl alcohol and stearyl alcohol), octyldodecanol, isostearyl alcohol, 2-hexyl decanol, palmityl alcohol, myristyl alcohol, stearyl alcohol, lauryl alcohol, oleic alcohol (or oleyl), linoleyl alcohol (or linoley-ether), linolenic alcohol (or linolenyl) and undecylenic alcohol, and mixtures thereof, and more preferably from cetyl alcohol, stearyl alcohol, and cetearyl alcohol, and mixtures thereof.

In one embodiment of the present invention, the at least one component (d) comprises polyols selected from ethylene glycol, propylene glycol, 1,3-butylene glycol, pentylene glycol, hexylene glycol, dipropylene glycol, glycerin, and mixtures thereof.

In one embodiment of the present invention, the at least one component (d) comprises fatty substances other than a fatty alcohol and selected from hydrocarbons, fatty esters, plant/vegetable oils, esters of fatty acid, alkanes, waxes, non-silicone synthetic oils, and mixtures thereof, and preferably selected from cetyl esters, mineral oil, paraffin oils, petroleum jelly, liquid paraffin, polydecenes, hydrogenated polyisobutene, perfluoromethylcyclopentane, perfluoro-1,3-dimethylcyclohexane, dodecafluoropentane, hexane, dodecane, isohexadecane, isodecane, pentaclethra macroloba seed oil, sunflower oil, maize oil, soya oil, cucurbit oil, grapeseed oil, sesame oil, hazelnut oil, apricot oil, macadamia oil, arara oil, sunflower oil, castor oil, avocado oil, jojoba oil, shea butter oil, and mixtures thereof.

In one embodiment of the present invention, the composition further comprises at least one neutralizing agent selected from organic amines, alkali metal hydroxides, alkali earth metal hydroxides, alkali metal carbonates, alkali metal phosphates, and mixtures thereof, and preferably selected from aminomethyl propanol, sodium hydroxide, potassium hydroxide, lithium hydroxide, aminomethyl propanediol, triisopropanol amine, dimethylstearylamine, dimethyl/tallowamine, lysine, ornithine, arginine, monoethanolamine, triethanolamine, calcium hydroxide, calcium bicarbonate, and mixtures thereof.

In one embodiment of the present invention, the composition further comprisies at least one amino silicone selected from amodimethicone, trideceth-9 PG amodimethicone, PEG-40/PPG-8 Methylaminopropyl/hydroxypropyl dimethicone copolymer, and mixtures thereof.

In one embodiment of the present invention, the composition further comprisies at least one cationic conditioning polymer selected from cationic cellulose derivatives, cationic gum derivatives, polymer derivatives of diallyldimethyl ammonium chloride, polymer derivatives of methacrylamidopropyltrimethylammonium chloride, and mixtures thereof and preferably from polyquaternium-7, polyquaternium-10, guar hydroxypropyltrimonium chloride, and mixtures thereof.

In one embodiment of the present invention, the pH of the compositions of the present invention ranges from about pH 2.5 to about 6.5, or from about pH 3 to about 6, or from about pH 3 to about 5.2, such as from about pH 3 to about 5, or preferably from about pH 3 to about 4.8, or more preferably from about pH 3 to about 4.5, or even more preferably from about pH 3 to about 4, including all ranges and sub ranges therebetween.

In one embodiment, the present invention is directed to a hair cosmetic composition comprising:

(a) at least one cationic surfactant, preferably selected from quaternary ammonium compounds and their salts, and more preferably selected from cetrimonium chloride, behentrimonium chloride, dipalmitoylethylhydroxyethylmonium methosulfate, and mixtures thereof and present in an amount of from about 0.7% to about 5% by weight;

(b) at least one thiol-based compound selected from thiolactic acid, thiolactic acid derivatives, their salts, and mixtures thereof and present in an amount of from about 0.7% to about 12% by weight;

(c) at least one fatty alcohol selected from cetyl alcohol, stearyl alcohol, cetearyl alcohol, and mixtures thereof and present in an amount of from about 7% to about 12% by weight;

(d) at least component selected from (i) polyols, (ii) fatty substances other than a fatty alcohol, and mixtures thereof in an amount of from about 0.3% to about 5% by weight;

(e) water; and

(f) optionally, at least one neutralizing agent;

wherein the pH of the composition ranges from about 3 to about 4;

all weights being relative to the total weight of the composition.

In one embodiment, the at least component (d) in the above-described composition comprises:

(i) polyols selected from ethylene glycol, propylene glycol, 1,3-butylene glycol, pentylene glycol, hexylene glycol, dipropylene glycol, glycerin, and mixtures thereof, and preferably, from glycerin; and/or

(ii) mineral oil, cetyl esters;

(iii) and mixtures thereof.

In one embodiment, the above-described composition further comprises:

(iv) at least one amino silicone selected from amodimethicone in an amount of from about 0.1% to about 2% by weight, relative to the total weight of the composition; and/or

(v) at least one cationic conditioning polymer selected from polyquaternium-7, polyquaternium-10, guar hydroxypropyltrimonium chloride, and mixtures thereof and present in an amount of from about 0.05% to about 2% by weight, relative to the total weight of the composition.

In one embodiment of the present invention, any one of the above-described compositions is substantially free of anionic surfactants selected from sulfate surfactants, sulfonate surfactants, sarcosinate surfactants, and mixtures thereof.

In one embodiment of the present invention, any one of the above-described compositions is a rinse-off conditioner or mask composition.

In one embodiment, the present invention is directed to a method of treating hair, the method comprising a treatment cycle involving the steps of:

a) optionally, washing/rinsing the hair with a shampoo having a neutral pH and/or rinsing the hair with water, followed by allowing the hair to air dry, while optionally applying a smoothing action on the hair;

b) applying the composition of any one of the preceding claims onto the hair;

c) allowing the composition to remain on the hair for a period of time ranging from 1 to 10 minutes or from 1 to 5 minutes;

d) rinsing the hair with water;

e) drying the hair at a temperature ranging from room temperature up to about 100° C., while optionally applying a smoothing action on the hair;

f) passing a flat iron over the hair swatch at least three times; and

g) optionally, washing/rinsing the hair with a shampoo having a neutral pH and/or rinsing the hair with water, followed by allowing the hair to air dry, while optionally applying a smoothing action on the hair.

In accordance with the present invention, Applicants have surprisingly and unexpectedly discovered that the above-described compositions which contain thiolactic acid in combination with certain surfactants and ingredients effectively condition the hair, produce good foaming properties, and at the same time, provide other hair care benefits to hair fibers such as straightening or texlaxing effects, volume reduction, frizz control, curl reduction, manageability, discipline, cosmeticity, and smooth feel.

It was also surprisingly and unexpectedly discovered that the application of the compositions of the present invention when used in combination with the processes of the present invention, results in effectively conditioned hair and imparts/improves the cosmetic properties of hair fibers, in particular human hair fibers such as the hair, for example, in terms of manageability, straightening or texlaxing effects, curl reduction, volume reduction, frizz control, manageability, discipline, cosmeticity, and smooth feel.

The compositions described above may be used on any type of hair, for example, light or dark hair, straight or curly, natural hair, or hair that has undergone a cosmetic treatment such as permanent waving, dyeing, bleaching or relaxing.

In a preferred embodiment, the composition of the present invention is applied on curly, embrittled, and/or damaged hair.

Other subjects and characteristics, aspects and advantages of the invention will emerge even more clearly on reading the description and the example that follows.

### Cationic Surfactant

The composition according to the invention comprises one or more cationic surfactants.

Non-limiting examples of cationic surfactants useful in the invention include, for example, optionally polyoxyalkylenated primary, secondary or tertiary fatty amine salts, quaternary ammonium salts, and mixtures thereof.

Quaternary ammonium salts useful in the invention include, for example:

quaternary ammonium salts having formula (Ia):

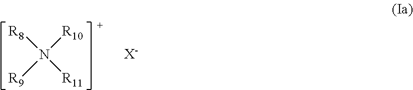

in which:

the groups R8 to R11, which may be identical or different, represent a linear or branched aliphatic group containing from 1 to 30 carbon atoms, or an aromatic group such as aryl or alkylaryl, at least one of the groups R8 to R11 containing from 8 to 30 carbon atoms and preferably from 12 to 24 carbon atoms; it being possible for the aliphatic groups to comprise heteroatoms such as, in particular, oxygen, nitrogen, sulfur or halogens;

The aliphatic groups are chosen, for example, from C1-C30 alkyl, C1-C30 alkoxy, (C2-C6)polyoxyalkylene, C1-C30 alkylamide, (C12-C22)alkyl(C2C6)alkylamido, (C12-C22)alkyl acetate and C1-C30 hydroxyalkyl groups.

X— is an anion chosen from the group consisting of halides, phosphates, acetates, lactates, (C1-C4)alkyl sulfates, (C1-C4)alkylsulfonates and (C1-C4)alkylarylsulfonates.

Among the quaternary ammonium salts having formula (Ia), preference is given to tetraalkylammonium chlorides such as, for example, dialkyldimethylammonium or alkyltrimethylammonium chlorides in which the alkyl group comprises approximately from 12 to 22 carbon atoms, particularly behenyltrimethylammonium, distearyldimethylammonium, cetyltrimethylammonium, and benzyldimethylstearylammonium chlorides, or secondly, to palmitylamidopropyltrimethylammonium chloride or stearamidopropyldimethyl-(myristyl acetate)-ammonium chloride, which is sold under the name Ceraphyl® 70 by the company Van Dyk.

imidazoline quaternary ammonium salts having formula (IIa):

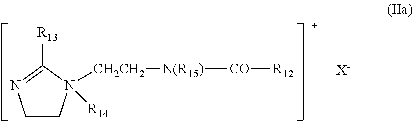

in which

R12 represents an alkenyl or alkyl group comprising from 8 to 30 carbon atoms, for example fatty acid derivatives of tallow;

R13 represents a hydrogen atom, a C1-C4 alkyl group or an alkenyl or alkyl group comprising from 8 to 30 carbon atoms;

R14 represents a C1-C4 alkyl group;

R15 represents a hydrogen atom or a C1-C4 alkyl group;

X— is an anion chosen from the group consisting of halides, phosphates, acetates, lactates, (C1-C4)alkyl sulfates, (C1-C4)alkylsulfonates and (C1-C4)alkylarylsulfonates;

R12 and R13 preferably denote a mixture of alkenyl or alkyl groups containing from 12 to 21 carbon atoms, for example fatty acid derivatives of tallow, R14 denotes a methyl group, and R15 denotes a hydrogen atom. A product of this kind is sold for example under the name Rewoquat® W 75 by the company Rewo.

quaternary di- or triammonium salts having formula (IIIa):

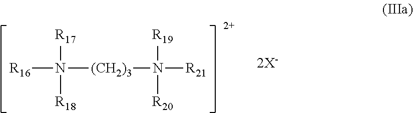

in which

R16 denotes an alkyl group containing approximately from 16 to 30 carbon atoms, which is optionally hydroxylated and/or interrupted with one or more oxygen atoms, R17 is chosen from hydrogen or an alkyl group containing from 1 to 4 carbon atoms or a group —(CH2)3-N+(R16a)(R17a)(R18a);

R16a, R17a, R18a, R18, R19, R20 and R21, which may be identical or different, are chosen from hydrogen and an alkyl group comprising from 1 to 4 carbon atoms; and

X— is an anion chosen from the group consisting of halides, acetates, phosphates, nitrates, (C1-C4)alkyl sulfates, (C1-C4)alkylsulfonates and (C1-C4)alkylarylsulfonates, in particular methyl sulfate and ethyl sulfate.

Such compounds are, for example, Finquat® CT-P, sold by the company Finetex (Quaternium 89), and Finquat® CT, sold by the company Finetex (Quaternium 75),

quaternary ammonium salts containing one or more ester functions having the following formula (IVa):

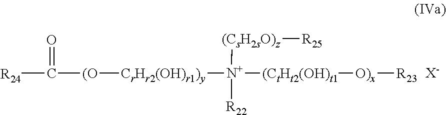

in which:

R22 is chosen from C1-C6 alkyl groups and C1-C6 hydroxyalkyl or C1-C6 dihydroxyalkyl groups;

R23 is chosen from the group R26-C(═O)—; hydrocarbon-based linear or branched, saturated or unsaturated C1-C22 groups R27; and a hydrogen atom;

R25 is chosen from the group R28-C(═O)—; hydrocarbon-based linear or branched, saturated or unsaturated C1-C6 groups R29; and a hydrogen atom;

R24, R26 and R28, which may be identical or different, are chosen from linear or branched, saturated or unsaturated C7-C21 hydrocarbon-based groups;

r, s and t, which may be identical or different, are integers ranging from 2 to 6;

r1 and t1, which may be identical or different, are equal to 0 or 1;

r2+r1=2 r and t1+t2=2 t;

y is an integer ranging from 1 to 10;

x and z, which may be identical or different, are integers ranging from 0 to 10;

X— is a simple or complex, organic or mineral anion;

with the proviso that the sum x+y+z is from 1 to 15, that when x is 0 then R23 denotes R27, and that when z is 0 then R25 denotes R29.

The alkyl groups R22 may be linear or branched, and more particularly linear. Preferably, R22 denotes a methyl, ethyl, hydroxyethyl or dihydroxypropyl group, and more particularly a methyl or ethyl group.

Advantageously, the sum x+y+z is from 1 to 10.

When R23 is an R27 hydrocarbon group, it may be long and may have from 12 to 22 carbon atoms, or may be short and may have from 1 to 3 carbon atoms.

When R25 is an R29 hydrocarbon group, it preferably has 1 to 3 carbon atoms.

Advantageously, R24, R26 and R28, which are identical or different, are chosen from linear or branched, saturated or unsaturated C11-C21 hydrocarbon groups, and more particularly from linear or branched, saturated or unsaturated C11-C21 alkyl and alkenyl groups.

Preferably, x and z, which may be identical or different, are equal to 0 or 1. Advantageously, y is equal to 1. Preferably, r, s and t, which may be identical or different, are equal to 2 or 3, and even more particularly are equal to 2.

The anion X— is preferably a halide, preferably chloride, bromide or iodide, a (C1-C4)alkyl sulfate, (C1-C4)alkyl sulfonate or (C1-C4)alkylaryl sulfonate. However, it is possible to use methanesulfonate, phosphate, nitrate, tosylate, an anion derived from an organic acid, such as acetate or lactate, or any other anion that is compatible with the ammonium comprising an ester function.

The anion X— is more particularly still chloride, methyl sulfate or ethyl sulfate.

Use is made more particularly, in the composition according to the invention, of the ammonium salts having formula (IVb) in which:

R22 denotes a methyl or ethyl group,

x and y are equal to 1,

z is equal to 0 or 1,

r, s and t are equal to 2,

R23 is chosen from the group R26-C(═O)—; methyl groups, ethyl groups or hydrocarbon-based C14-C22 groups; and a hydrogen atom,

R25 is chosen from the group R28-C(═O)—; and a hydrogen atom,

R24, R26 and R28, which may be identical or different, are chosen from linear or branched, saturated or unsaturated C13-C17 hydrocarbon groups, and preferably from linear or branched, saturated or unsaturated C13-C17 alkyl and alkenyl groups.

The hydrocarbon-based groups are advantageously linear.

Among the compounds of formula (IVb), examples that may be mentioned include salts, in particular the chloride or methyl sulfate of diacyloxyethyldimethylammonium, diacyloxyethylhydroxyethylmethylammonium, monoacyloxyethyldihydroxyethylmethylammonium, triacyloxyethylmethylammonium or monoacyloxyethylhydroxyethyldimethylammonium, and mixtures thereof. The acyl groups preferably contain 14 to 18 carbon atoms and are obtained more particularly from a plant oil, such as palm oil or sunflower oil. When the compound contains several acyl groups, these groups may be identical or different.

These products are obtained, for example, by direct esterification of triethanolamine, triisopropanolamine, alkyldiethanolamine or alkyldiisopropanol-amine, which are optionally oxyalkylenated, with fatty acids or with fatty acid mixtures of plant or animal origin, or by transesterification of the methyl esters thereof. This esterification is followed by a quaternization by means of an alkylating agent, such as an alkyl halide, preferably methyl or ethyl halide, a dialkyl sulfate, preferably methyl or ethyl sulfate, methyl methanesulfonate, methyl para-toluenesulfonate, glycol chlorohydrin or glycerol chlorohydrin. Such compounds are, for example, sold under the names Dehyquart® by the company Henkel, Stepanquat® by the company Stepan, Noxamium® by the company Ceca or Rewoquat® WE 18 by the company Rewo-Witco.

The composition according to the invention may contain, for example, a mixture of quaternary ammonium monoester, diester and triester salts with a weight majority of diester salts. Use may also be made of the ammonium salts containing at least one ester function that are described in patents U.S. Pat. No. 4,874,554 and U.S. Pat. No. 4,137,180. Use may also be made of behenoylhydroxypropyltrimethylammonium chloride, for example, sold by the company Kao under the name Quartamin BTC 131.

Preferably, the ammonium salts containing at least one ester function.

Preferably, the cationic surfactants are chosen from cetrimonim chloride (cetyltrimethylammonium salt), behentrimonim chloride (behenyltrimethylammonium salt), behentrimonium methosulfate, dipalmitoylethylhydroxyethylmethylammonium salts, and mixtures thereof.

The composition according to the invention comprise the cationic surfactant in an amount ranging from about 0.1% to about 10% by weight, preferably from about 0.5% to about 8% by weight, and most preferably from about 0.7% to about 5% by weight, relative to the total weight of the composition, including all ranges and subranges therebetween.

In a particular embodiment, the amount of the at least one cationic surfactant is at about 0.1%, 0.125%, 0.15%, 0.2%, 0.25% 0.3%, 0.325%, 0.35%, 0.375%, 0.4%, 0.425%, 0.45%, 0.5%, 0.55%, 0.6%, 0.65%, 0.7%, 0.75%, 0.8%, 0.85%, 0.9%, 0.95%, 1%, 1.25%, 1.5%, 1.75%, 2%, 2.25%, 2.4%, 2.5%, 2.75%, 3%, 3.25%, 3.5%, 3.75%, 4%, 4.25%, 4.5%, 4.75%, or 5% by weight, relative to the total weight of the composition.

### Thiol-Based Compounds

The present invention employs at least one thiol-based compound selected from thiolactic acid, thiolactic acid derivatives, their salts, and mixtures thereof.

The at least one thiol-based compound of the present disclosure can be used in combination with other thiol-based compounds selected from thioglycolic acid, cysteine, cysteamine, homocystine, glutathione, thioglycerol, thiomalic acid, 2-mercaptopropionic acid, 3-mercaptopropionic acid, thio diglyco I, 2-mercaptoethanol, dithiothreitol, thioxanthine, thiosalicylic acid, thiopropionic acid, lipoic acid, N-acetylcysteine, their salts thereof, and mixtures thereof.

The at least one thiol-based compound of the present disclosure can be also be used in combination with non-thiol based compounds such as alkali metal, alkaline-earth metal sulfites, hydrides or phosphines, and mixtures thereof.

In certain embodiments, the thiol-based compound used in the composition of the invention is thiolactic acid.

The at least one thiol-based compound can be employed in the compositions of the present invention in an amount of from about 0.5% to about 15% by weight, preferably from about 0.6% to about 14% by weight, more preferably from about 7% to about 12% by weight, more preferably from about 0.8% to about 10% by weight, or from about 1% to about 8% by weight, relative to the total weight of the composition, including all ranges and subranges therebetween.

In certain embodiments, the at least one thiol-based compound is selected from thiolactic acid and is employed in the composition of the present invention in an amount of about 1%, 1.25%, 1.5%, 1.75%, 2%, 2.25%, 2.5%, 2.75%, 3%, 3.25%, 3.5%, 3.75%, 4%, 4.25%, 4..5%, 4.75%, 5%, 5.25%, 5.5%, 5.75%, 6%, 6.25%, 6.5%, 6.75%, 7%, 7.25%, 7.5%, 7.75%, 8%, 8.25%, 8.5%, 8.75%, 9%, 9.5%, 10%, 10.5%, 11%, 11.5%, 12%, 13%, 14% by weight, based on the total weight of the composition.

### Fatty Alcohol

The fatty alcohols correspond to linear, branched saturated/unsaturated fatty alcohols comprising from 6 to 60 carbon atoms and preferably correspond to the formula R—OH in which R is a saturated or unsaturated, linear or branched hydrocarbon-based radical, comprising 6 to 60 carbon atoms, or from 10 to 50 carbon atoms, or from 12 to 24 carbon atoms, or from 10 to 22 carbon atoms optionally comprising one or more OH groups.

The saturated fatty alcohols are preferably branched and can be in liquid form. They can optionally comprise, in their structure, at least one aromatic or non-aromatic ring. They are preferably acyclic.

The unsaturated fatty alcohols exhibit, in their structure, at least one double or triple bond and preferably one or more double bonds. When several double bonds are present, there are preferably 2 or 3 of them and they can be conjugated or unconjugated. These unsaturated fatty alcohols can be linear or branched. They can optionally comprise, in their structure, at least one aromatic or non-aromatic ring. They are preferably acyclic.

Liquid fatty alcohols may be selected, for example, from octyldodecanol, 2-butyloctanol, 2-hexyldecanol, 2-undecylpentadecanol, oleyl alcohol, linoleyl alcohol, isostearyl alcohol, undecylenyl alcohol, linolenyl alcohol and mixtures thereof.

The fatty alcohols of the invention may be in solid form and may be non-oxyalkylenated and/or non-glycerolated. These fatty alcohols may be constituents of animal or plant waxes.

The solid fatty alcohol may represent a mixture of fatty alcohols, which means that several species of fatty alcohol may coexist, in the form of a mixture, in a commercial product. One example of such a commercial product is cetearyl alcohol, a mixture of cetyl alcohol and stearyl alcohol, commercially available under the trade name of LANETTE-O from the company BASF. Cetyl alcohol may also be commercially available under the tradename of LANETTE 16 from the company BASF.

In an embodiment, the solid fatty alcohols of the present invention may be chosen from myristyl alcohol, cetyl alcohol, stearyl alcohol, cetearyl alcohol, and mixtures thereof, octyldodecanol, 2-butyloctanol, 2-hexyldecanol, 2-undecyl-pentadecanol, oleic alcohol, linoleic alcohol, behenyl alcohol, and mixtures thereof.

Other suitable examples of the solid fatty alcohol of the present invention include branched solid fatty alcohols chosen from 2-dodecylhexadecanol, 2-tetradecyl-1-octadecanol, 2-tetradecyl-1-eicosanol, 2-hexadecyl-1-octadecanol and 2-hexadecyl-1-eicosanol, and mixtures thereof.

In one embodiment, the at least one fatty alcohol is selected from linear saturated/unsaturated fatty alcohols comprising from 6 to 60 carbon atoms and preferably from cetyl alcohol, stearyl alcohol, cetearyl alcohol (mixture of cetyl alcohol and stearyl alcohol), octyldodecanol, isostearyl alcohol, 2-hexyl decanol, palmityl alcohol, myristyl alcohol, stearyl alcohol, lauryl alcohol, oleic alcohol (or oleyl), linoleyl alcohol (or linoley-ether), linolenic alcohol (or linolenyl) and undecylenic alcohol, and mixtures thereof.

In embodiment of the present invention, the fatty alcohol comprises cetyl alcohol and stearyl alcohol or cetearyl alcohol.

In embodiment of the present invention, the fatty alcohol is chosen from cetyl alcohol, stearyl alcohol, cetearyl alcohol, and mixtures thereof.

The composition according to the invention comprises the fatty alcohol in an amount of from about 6% to about 20% by weight, or from about 6.5% to about 15% by weight, or preferably from about 7% to about 12% by weight, relative to the total weight of the composition, including all ranges and subranges therebetween.

In a particular embodiment, the total amount of fatty alcohols is at about 6%, 6.1, 6.25%, 6.4%, 6.5%, 6.6%, 6.75%, 6.9%, 7%, 7.1%, 7.25%, 7.4%, 7.5%, 7.6%, 7.75%, 7.9%, 8%, 8.1%, 8.2%, 8.25%, 8.4%, 8.5%, 8.6%, 8.75%, 8.9%, 9%, 9.1%, 9.2%, 9.25%, 9.4%, 9.5%, 9.6%, 9.75%, 9.9%, 10%, 10.1%, 10.2%, 10.25%, 10.4%, 10.5%, 10.6%, 10.75%, 10.9%, 11%, 11.25%, 11.5%. 11.75%, or 12% by weight, relative to the total weight of the composition.

### Component Selected from Polyols and Fatty Substances Other than a Fatty Alcohol

The compositions of the present invention also comprise at least one component selected from (i) polyols, (ii) fatty substances other than a fatty alcohol, and mixtures thereof.

(i) Polyols

The polyols of the present invention may be selected from compounds containing from 2 to 8 carbon atoms, such as ethylene glycol, propylene glycol, 1,3-butylene glycol, pentylene glycol, hexylene glycol, dipropylene glycol, glycerin, and mixtures thereof.

In one embodiment, the polyols of the present invention are selected from glycerin, propylene glycol, and mixtures thereof.

In one embodiment, the polyol of the present invention is glycerin.

(ii) Fatty substances other than a fatty alcohol

The fatty substances other than a fatty alcohol in the present invention may be selected from hydrocarbons, fatty esters, plant/vegetable oils, esters of fatty acid, alkanes, waxes, non-silicone synthetic oils, and mixtures thereof, and preferably selected from cetyl esters, mineral oil, paraffin oils, petroleum jelly, liquid paraffin, polydecenes, hydrogenated polyisobutene, perfluoromethylcyclopentane, perfluoro-1,3-dimethylcyclohexane, dodecafluoropentane, hexane, dodecane, isohexadecane, isodecane, pentaclethra macroloba seed oil, sunflower oil, maize oil, soya oil, cucurbit oil, grapeseed oil, sesame oil, hazelnut oil, apricot oil, macadamia oil, arara oil, sunflower oil, castor oil, avocado oil, jojoba oil, shea butter oil, and mixtures thereof.

In one embodiment, the fatty substances other than a fatty alcohol in the present invention may be selected from mineral oil, cetyl esters, and mixtures thereof.

In one embodiment, the at least one component (d) of the present invention comprises (i) polyols and/or (ii) fatty substances other than a fatty alcohol.

In accordance with the present invention, the at least one component (d) is present in an amount of from about 0.1% to about 10% by weight, preferably from about 0.2% to about 8% by weight, and more preferably from about 0.3% to about 5% by weight, relative to the total weight of the composition, including all ranges and subranges therebetween.

In a particular embodiment, the total amount of the at least one component (d) is at about 0.1%, 0.15%, 0.2%, 0.25%, 0.3%, 0.325%, 0.35%, 0.375%, 0.4%, 0.425%, 0.45%, 0.475%, 0.5%, 0.525%, 0.55%, 0.575%, 0.6, 0.625%, 0.65%, 0.675%, 0.7%, 0.725%, 0.75%, 0.775%, 0.8%, 0.825%, 0.85%, 0.875%, 0.9%, 0.925%, 0.95%, 0.975%, 1%, 1.25%, 1.5%, 1.55%, 1.6%, 1.65%, 1.7%, 1.75%, 1.8%, 1.85%, 1.9%, 1.95%, 2%, 2.05%, 2.1%, 0, 2.15%, 2.2%, 2.25%, 2.3%, 0, 2.35%, 2.4%, 2.45%, 2.5%, 2.55%, 2.6%, 2.65%, 2.7%, 2.75%, 2.8%, 2.85%, 2.9%, 3.0%, 3.25%, 3.5%, 3.75%, 4%, 4.25%, 4.5%, 4.75%, or 5% by weight, relative to the total weight of the composition.

### Cationic Conditioning Polymer

The composition according to the invention may further comprise one or more cationic polymers. The cationic conditioning polymer used in the invention comprises homopolymers, copolymers, and mixtures thereof.

Non-limiting examples of cationic conditioning polymers useful in the invention include, for example, cationic cellulose derivatives, such as for example polyquaternium-10; cationic gum derivatives such as for example gum derivatives, including particularly guar hydroxypropyltrimonium chloride; polymer derivatives of diallyldimethyl ammonium chloride (“poly-DADMAs”) and of methacrylamido-propyltrimethylammonium chloride (“poly-MAPTACs”), and mixtures thereof

Non-limiting examples of poly-DADMAs and poly-poly-MAPTACs include, polyquaternium-4, polyquaternium-5, polyquaternium-6, polyquaternium-7, polyquaternium-22, polyquaternium-37, polyquaternium-39, polyquaternium-47, polyquaternium-53, and mixtures thereof.

The composition according to the invention preferably comprises cationic polymers known under the INCI names polyquaternium-7, guar hydroxypropyltrimonium chloride, and mixtures thereof.

The cationic polymer is in an amount ranging from about 0.01% to about 4% by weight, preferably from about 0.05% to about 3% by weight, and most preferably from about 0.05% to about 2% by weight, relative to the total weight of the composition.

### Amino Silicones

The cosmetic composition according to the invention optionally may comprise one or more amino silicones. The term “amino silicone” is intended to mean any silicone comprising at least one primary, secondary or tertiary amine or a quaternary ammonium group.

As amino silicone that may be used in the scope of the invention, the following can be cited:

a) polysiloxanes corresponding to formula (A):

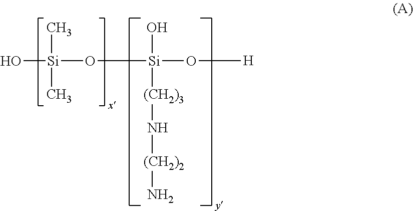

in which x′ and y′ are integers such that the weight-average molecular weight (Mw) is comprised between about 5000 and 500 000;

b) amino silicones corresponding to formula (B):

R′aG3-a-Si(OSiG2)n-(OSiGbR′2-b)m-O—SiG3-a-R′a(B)

in which:

G, which may be identical or different, designate a hydrogen atom, or a phenyl, OH or C1-C8 alkyl group, for example methyl, or C1-C8 alkoxy, for example methoxy,

a, which may be identical or different, denote the number 0 or an integer from 1 to 3, in particular 0;

b denotes 0 or 1, and in particular 1;

m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;

R′, which may be identical or different, denote a monovalent radical having formula -CqH2qL in which q is a number ranging from 2 to 8 and L is an optionally quaternized amino group chosen from the following groups:

—NR″-Q-N(R″)2

—N(R″)2

—N+(R″)3 A-

—N+H(R″)2 A-

—N+H2(R″) A-

—N(R″)-Q-N+R″H2 A-

—NR″-Q-N+(R″)2H A-

—NR″-Q-N+(R″)3 A-,

in which R″, which may be identical or different, denote hydrogen, phenyl, benzyl, or a saturated monovalent hydrocarbon-based radical, for example a C1-C20 alkyl radical; Q denotes a linear or branched CrH2r group, r being an integer ranging from 2 to 6, preferably from 2 to 4; and A- represents a cosmetically acceptable ion, in particular a halide such as fluoride, chloride, bromide or iodide.

A group of amino silicones corresponding to this definition (B) is represented by the silicones called “trimethylsilylamodimethicone” having formula (C):

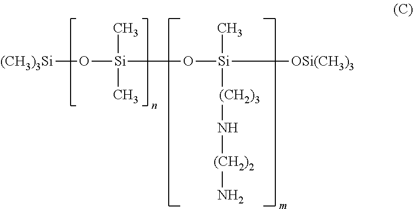

in which n and m have the meanings given above, in formula B.

Another group of amino silicones corresponding to this definition is represented by silicones having the following formulae (D) or (E):

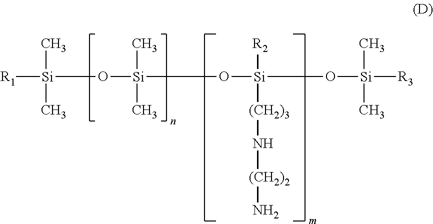

in which:

m and n are numbers such that the sum (n+m) can range from 1 to 1000, in particular from 50 to 250 and more particularly from 100 to 200, it being possible for n to denote a number from 0 to 999 and in particular from 49 to 249, and more particularly from 125 to 175, and for m to denote a number from 1 to 1000 and in particular from 1 to 10, and more particularly from 1 to 5;

R1, R2, R3, which may be identical or different, represent a hydroxy or C1-C4 alkoxy radical, where at least one of the radicals R1 to R3 denotes an alkoxy radical, preferably a methoxy radical.

The hydroxy/alkoxy mole ratio ranges preferably from 0.2:1 to 0.4:1 and preferably from 0.25:1 to 0.35:1 and more particularly equals 0.3:1.

The weight-average molecular weight (Mw) of the silicone ranges preferably from 2000 to 1 000 000, more particularly from 3500 to 200 000.

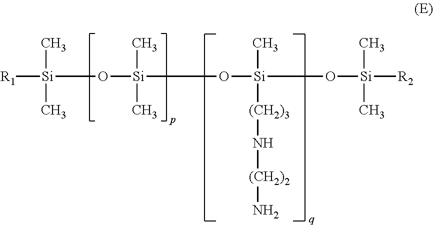

in which:

p and q are numbers such that the sum (p+q) ranges from 1 to 1000, particularly from 50 to 350, and more particularly from 150 to 250; it being possible for p to denote a number from 0 to 999 and in particular from 49 to 349, and more particularly from 159 to 239 and for q to denote a number from 1 to 1000, in particular from 1 to 10, and more particularly from 1 to 5;

R1, R2, which are different, represent a hydroxy or C1-C4 alkoxy radical, where at least one of the radicals R1 or R2 denotes an alkoxy radical, preferably a methoxy radical.

The hydroxy/alkoxy mole ratio ranges generally from 1:0.8 to 1:1.1 and preferably from 1:0.9 to 1:1 and more particularly equals 1:0.95.

The weight-average molecular weight (Mw) of the silicone ranges preferably from 2000 to 200 000, even more particularly 5000 to 100 000 and more particularly from 10 000 to 50 000.

Commercial products corresponding to these silicones having structure (D) or (E) may include in their composition one or more other amino silicones whose structure is different than formulae (D) or (E).

A product containing amino silicones having structure (D) is sold by Wacker under the name Belsil® ADM 652.

A product containing amino silicones having structure (E) is sold by Wacker under the name Fluid WR 1300®.

When these amino silicones are used, one particularly advantageous embodiment consists in using them in the form of an oil-in-water emulsion. The oil-in-water emulsion may comprise one or more surfactants. The surfactants may be of any nature but are preferably cationic and/or nonionic. The number-average size of the silicone particles in the emulsion generally ranges from 3 nm to 500 nanometres. Preferably, in particular as amino silicones having formula (E), microemulsions are used whose average particle size ranges from 5 nm to 60 nanometres (limits included) and more preferably from 10 nm to 50 nanometres (limits included). Accordingly, according to the invention the microemulsions of amino silicone having formula (E) sold as Finish CT 96 E® or SLM 28020® by Wacker can be used.

Another group of amino silicones corresponding to this definition is represented by the following formula (F):

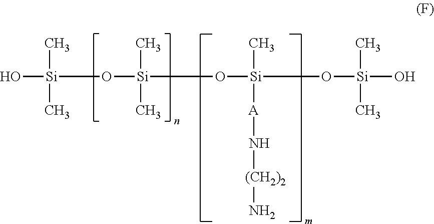

in which:

m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;

A denotes a linear or branched alkylene radical containing from 4 to 8 carbon atoms and preferably 4 carbon atoms. This radical is preferably linear.

The weight-average molecular weight (Mw) of these amino silicones ranges preferably from 2000 to 1 000 000 and even more particularly from 3500 to 200 000.

A preferred silicone of formula (F) is amodimethicone (INCI name) sold under the tradename XIAMETER® MEM-8299 Cationic Emulsion by Dow Corning.

Another group of amino silicones corresponding to this definition is represented by the following formula (G):

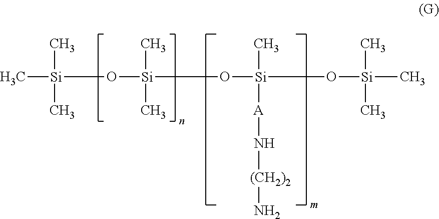

in which:

m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;

A denotes a linear or branched alkylene radical containing from 4 to 8 carbon atoms and preferably 4 carbon atoms. This radical is preferably branched.

The weight-average molecular weight (Mw) of these amino silicones ranges preferably from 500 to 1 000 000 and even more particularly from 1000 to 200 000.

A silicone having this formula is for example DC2-8566 Amino Fluid by Dow Corning.

c) amino silicones corresponding to formula (H):

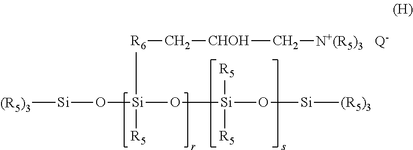

in which:

R5 represents a monovalent hydrocarbon-based radical containing from 1 to 18 carbon atoms, and in particular a C1-C18 alkyl or C2-C18 alkenyl radical, for example methyl;

R6 represents a divalent hydrocarbon-based radical, in particular a C1-C18 alkylene radical or a divalent C1-C18, for example C1-C8, alkylenoxy radical linked to the Si via an SiC bond;

Q- is an anion such as a halide ion, in particular chloride, or an organic acid salt (for example acetate);

r represents a mean statistical value from 2 to 20 and in particular from 2 to 8;

s represents a mean statistical value from 20 to 200 and in particular from 20 to 50.

Such amino silicones are described more particularly in patent U.S. Pat. No. 4,185,087.

d) quaternary ammonium silicones having formula (I):

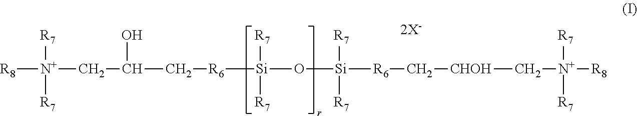

in which:

R7, which may be identical or different, represent a monovalent hydrocarbon-based radical containing from 1 to 18 carbon atoms, and in particular a C1-C18 alkyl radical, a C2-C18 alkenyl radical or a ring containing 5 or 6 carbon atoms, for example methyl;

R6 represents a divalent hydrocarbon-based radical, in particular a C1-C18 alkylene radical or a divalent C1-C18, for example C1-C8, alkylenoxy radical linked to the Si via an SiC bond;

R8, which may be identical or different, represent a hydrogen atom, a monovalent hydrocarbon-based radical containing from 1 to 18 carbon atoms, and in particular a C1-C18 alkyl radical, a C2-C18 alkenyl radical or a —R6-NHCOR7 radical;

X— is an anion such as a halide ion, in particular chloride, or an organic acid salt (for example acetate);

r represents a mean statistical value from 2 to 200 and in particular from 5 to 100;

These silicones are described, for example, in patent application EP-A 0 530 974.

e) amino silicones having formula (J):

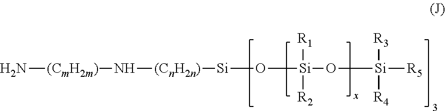

in which:

R1, R2, R3 and R4, which may be identical or different, denote a C1-C4 alkyl radical or a phenyl group;

R5 denotes a C1-C4 alkyl radical or a hydroxyl group;

n is an integer ranging from 1 to 5;

m is an integer ranging from 1 to 5;

and in which x is chosen such that the amine number is between 0.01 and 1 meq/g;

f) multiblock polyoxyalkylenated amino silicones, of type (AB)n, A being a polysiloxane block and B being a polyoxyalkylenated block containing at least one amine group.

Said silicones are preferably constituted of repeating units having the following general formulae:

[—(SiMe2O)xSiMe2-R—N(R″)—R′—O(C2H4O)a(C3H6O)b-R′—N(H)—R—]

or alternatively

[—(SiMe2O)xSiMe2-R—N(R″)—R′—O(C2H4O)a(C3H6O)b-]

in which:

a is an integer greater than or equal to 1, preferably ranging from 5 to 200, more particularly ranging from 10 to 100;

b is an integer comprised between 0 and 200, preferably ranging from 4 to 100, more particularly between from 5 and 30;

x is an integer ranging from 1 to 10 000, more particularly from 10 to 5000;

R″ is a hydrogen atom or a methyl;

R, which may be identical or different, represent a divalent linear or branched C2-C12 hydrocarbon-based radical, optionally including one or more heteroatoms such as oxygen; preferably, R denotes an ethylene radical, a linear or branched propylene radical, a linear or branched butylene radical, or a —CH2CH2CH2OCH(OH)CH2— radical; preferentially R denotes a —CH2CH2CH2OCH(OH)CH2— radical;

R′, which may be identical or different, represent a divalent linear or branched C2-C12 hydrocarbon-based radical, optionally including one or more heteroatoms such as oxygen; preferably, R′ denotes an ethylene radical, a linear or branched propylene radical, a linear or branched butylene radical, or a —CH2CH2CH2OCH(OH)CH2— radical; preferentially R′ denotes —CH(CH3)—CH2-.

The siloxane blocks preferably represent between 50 and 95 mol % of the total weight of the silicone, more particularly from 70 to 85 mol %.

The amine content is preferably between 0.02 and 0.5 meq/g of copolymer in a 30% solution in dipropylene glycol, more particularly between 0.05 and 0.2.

The weight-average molecular weight (Mw) of the silicone is preferably comprised between 5000 and 1 000 000, more particularly between 10 000 and 200 000.

Mention may be made especially of the silicones sold under the names Silsoft™ A-843 or Silsoft™ A+ by Momentive.

g) the alkylamino silicones corresponding to formula (K) below:

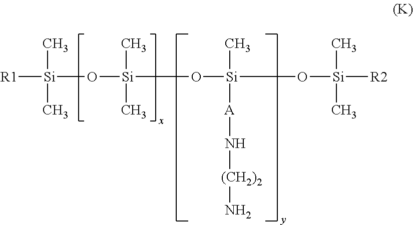

in which:

x and y are numbers ranging from 1 to 5000; preferably, x ranges from 10 to 2000 and especially from 100 to 1000; preferably, y ranges from 1 to 100;

R1 and R2, which may be identical or different, preferably identical, are linear or branched, saturated or unsaturated alkyl radicals, comprising 6 to 30 carbon atoms, preferably 8 to 24 carbon atoms and especially 12 to 20 carbon atoms;

A denotes a linear or branched alkylene radical containing from 2 to 8 carbon atoms,

Preferably, A comprises 3 to 6 carbon atoms, especially 4 carbon atoms; preferably, A is branched. Mention may be made especially of the following divalent radicals: —CH2CH2CH2 and —CH2CH(CH3)CH2-.

Preferably, R1 and R2, which may be identical or different, are saturated linear alkyl radicals comprising 6 to 30 carbon atoms, preferably 8 to 24 carbon atoms and especially 12 to 20 carbon atoms; mention may be made in particular of dodecyl, tetradecyl, pentadecyl, hexadecyl, heptadecyl, octadecyl, nonadecyl and eicosyl radicals; and preferentially, R1 and R2, which may be identical or different, are chosen from hexadecyl (cetyl) and octadecyl (stearyl) radicals.

Preferentially, the silicone is of formula (K) with:

x ranging from 10 to 2000 and especially from 100 to 1000;

y ranging from 1 to 100;

A comprising 3 to 6 carbon atoms and especially 4 carbon atoms; preferably, A is branched; and more particularly A is chosen from the following divalent radicals: —CH2CH2CH2 and —CH2CH(CH3)CH2-; and

R1 and R2, which may be identical or different, being linear, saturated alkyl radicals comprising 6 to 30 carbon atoms, preferably 8 to 24 carbon atoms and especially 12 to 20 carbon atoms; chosen in particular from dodecyl, tetradecyl, pentadecyl, hexadecyl, heptadecyl, octadecyl, nonadecyl and eicosyl radicals; preferentially, R1 and R2, which may be identical or different, being chosen from hexadecyl (cetyl) and octadecyl (stearyl) radicals.

A preferred silicone of formula (K) is bis-cetearyl amodimethicone (INCI name). Mention may be made especially of the silicone sold under the name Silsoft™ AX by Momentive.

In one embodiment, the amino silicones according to the invention are chosen from amodimethicone, trideceth-9 PG amodimethicone, PEG-40/PPG-8 Methylaminopropyl/hydroxypropyl dimethicone copolymer, and mixtures thereof.

Preferably, the amino silicones according to the invention are chosen from the amino silicones of formula (F). A preferred silicone of formula (F) is amodimethicone (INCI name) sold under the tradename XIAMETER® MEM-8299 Cationic Emulsion by Dow Corning.

The hair at least one silicone in the cosmetic composition according to the present invention is present in an amount of from about 0.01% to about 5% by weight, preferably from about 0.05% to about 3% by weight, and most preferably from about 0.1% to about 2% by weight, relative to the total weight of the composition, including all ranges and sub ranges therebetween.

### Neutralizing Agents

The compositions of the present invention may further comprise at least one neutralizing agent wherein the at least one neutralizing agent is selected from organic amines, alkali metal hydroxides, alkali earth metal hydroxides, alkali metal carbonates, alkali metal phosphates, and mixtures thereof,

Organic amines may be chosen from organic amines comprising one or two primary, secondary, or tertiary amine functions, and at least one linear or branched C1-C8 alkyl groups bearing at least one hydroxyl radical.

Organic amines may also be selected cyclic amines and other cyclic compounds, saturated or unsaturated, having one or more nitrogen atoms within the ring, and mixtures thereof.

The organic amines may be chosen from the ones having a pKb at 25° C. of less than 12, such as less than 10 or such as less than 6. It should be noted that this is the pKb corresponding to the function of highest basicity.

Organic amines may also be chosen from alkanolamines such as mono-, di- or trialkanolamines, comprising one to three identical or different C1-C4 hydroxyalkyl radicals, ethylamines, ethyleneamines, quinoline, aniline and cyclic amines, such as pyrroline, pyrrole, pyrrolidine, imidazole, imidazolidine, imidazolidinine, morpholine, pyridine, piperidine, pyrimidine, piperazine, triazine and derivatives thereof.

Among the compounds of the alkanolamine type that may be mentioned include but not limited to: monoethanolamine (also known as monoethanolamine or MEA), diethanolamine, triethanolamine, monoisopropanolamine, diisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol, 2-amino-2-methyl-1-propanol (aminomethyl propanol), and tris(hydroxymethylamino)methane.

Other examples include but are not limited to: 1,3-diaminopropane, 1,3-diamino-2-propanol, spermine, and spermidine.

In some embodiments, the organic amines are chosen from amino acids.

As non-limiting examples, the amino acids that may be used may be of natural or synthetic origin, in L, D, or racemic form, and comprise at least one acid function chosen from, for instance, carboxylic acid, sulfonic acid, phosphonic acid, and phosphoric acid functions. The amino acids may be in their neutral or ionic form.

Further as non-limiting examples, the amino acids may be chosen from basic amino acids comprising an additional amine function optionally included in a ring or in a ureido function.

Amino acids that may be used in the present disclosure include but are not limited to: aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine, ornithine, citrulline, and valine.

In some embodiments, the organic amines are chosen from organic amines of heterocyclic type. Besides histidine that has already been mentioned in the amino acids, non-limiting mention may also be made of pyridine, piperidine, imidazole, 1,2,4-triazole, tetrazole, and benzimidazole.

In some embodiments, the organic amines are chosen from amino acid dipeptides. Amino acid dipeptides that may be used in the present disclosure include but not limited to: carnosine, anserine, and baleine.

In some embodiments, the organic amines are chosen from compounds comprising a guanidine function. Organic amines of this type that may be used in the present disclosure include, besides arginine that has already been mentioned as an amino acid, creatine, creatinine, 1,1-dimethylguanidine, 1,1-diethylguanidine, glycocyamine, metformin, agmatine, N-amidinoalanine, 3-guanidinopropionic acid, 4-guanidinobutyric acid, and 2-([amino(imino)methyl]amino)ethane-1-sulfonic acid.

The alkali metal phosphates and carbonates that may be used are, for example, sodium phosphate, potassium phosphate, sodium carbonate, sodium bicarbonate, potassium carbonate, potassium bicarbonate, and their derivatives.

Neutralizing agents may also be chosen from alkali metal hydroxides, alkaline-earth metal hydroxides, transition metal hydroxides, quaternary ammonium hydroxides, organic hydroxides, and mixtures thereof. Suitable examples are ammonium hydroxide, sodium hydroxide, potassium hydroxide, lithium hydroxide, rubidium hydroxide, caesium hydroxide, francium hydroxide, beryllium hydroxide, magnesium hydroxide, calcium hydroxide, strontium hydroxide, barium hydroxide, molybdenum hydroxide, manganese hydroxide, zinc hydroxide, cobalt hydroxide, cadmium hydroxide, cerium hydroxide, lanthanum hydroxide, actinium hydroxide, thorium hydroxide, aluminium hydroxide, guanidinium hydroxide and mixtures thereof.

According to at least one embodiment, the at least one neutralizing agent is chosen from aminomethyl propanol, sodium hydroxide, potassium hydroxide, lithium hydroxide, aminomethyl propanediol, triisopropanol amine, dimethyl-stearylamine, dimethyl/tallowamine, lysine, ornithine, arginine, monoethanolamine, triethanolamine, calcium hydroxide, calcium bicarbonate, and mixtures thereof.

According to another preferred embodiment, the at least one neutralizing agent is chosen from aminomethyl propanol, sodium hydroxide, lithium hydroxide, calcium hydroxide, monoethanolamine, and mixtures thereof.

The at least neutralizing agent may be present in an amount of from 0.01% to 3% by weight, preferably from 0.02% to 2.75% by weight, more preferably from 0.025% to 2.5% by weight, even more preferably from 0.03% to 2% by weight, relative to the total weight of the composition, including all ranges and sub ranges therebetween.

When the at least neutralizing agent is selected from aminomethyl propanol, it is present in an amount of from 0.1% to 6.3% by weight, preferably from 0.2% to 5.5% by weight, more preferably from 0.3% to 5% by weight, even more preferably from 0.3% to 4.6% by weight, relative to the total weight of the composition, including all ranges and sub ranges therebetween.

When the at least neutralizing agent is selected from sodium hydroxide, it is present in an amount of from 0.1% to 4.1% by weight, preferably from 0.15% to 3.5% by weight, more preferably from 0.2% to 3% by weight, even more preferably from 1% to 3% by weight, relative to the total weight of the composition, including all ranges and sub ranges therebetween.

When the at least neutralizing agent is selected from monoethanolamine, it is present in an amount of from 0.1% to 4.1% by weight, preferably from 0.15% to 3.5% by weight, more preferably from 0.2% to 3% by weight, even more preferably from 1% to 3% by weight, relative to the total weight of the composition, including all ranges and sub ranges therebetween.

### pH

The pH of the compositions of the present invention may range from about 2 to less than 7, or from about pH 2.5 to about 6.5, or from about pH 3 to about 6, or from about pH 3 to about 5.2, such as from about pH 3 to about 5, or preferably from about pH 3 to about 4.8, or more preferably from about pH 3 to about 4.5, or even more preferably from about pH 3 to about 4, including all ranges and sub ranges therebetween.

In some embodiments, the pH of the compositions of the present invention can be at about 2, 2.5, 2.75, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5.

The desired pH may be obtained by using one or more of the neutralizing agents described above.

### Water

The composition according to the invention comprises water. The water used may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene. Water may also comprise reconstituted thermal water, that is to say a water comprising trace elements such as zinc, copper, magnesium, etc., reconstituting the characteristics of a thermal water.

The composition according to the invention comprises water in an amount ranging from about 50% to about 99% by weight, preferably from about 65% to about 95% by weight, and most preferably from about 70% to about 92% by weight, relative to the total weight of the composition.

### Organic Solvent (Optional)

The compositions according to the invention optionally may comprise one or more organic solvent.

Non-limiting examples of organic solvents useful in the invention include, for example, lower carbon alcohols such as ethanol, propanol, isopropanol, and mixtures thereof.

When present in the composition according to the invention, the water-soluble solvent is preferably present in the composition according to the invention in an amount of from about 0.01% to 10% by weight, preferably in an amount of from about 0.05% to 5% by weight, and most preferably 0.1% to 1% by weight, based on the total weight of the composition.

The composition according to the invention may also comprise at least one additional component selected from organic solvents, thickening agents (or rheology-/viscosity-modifying agents), silicones other than amino functional silicones, plasticizers, opacifiers, plant/vegetable oils, hydrocarbons, lower alkanes, fragrance, preservatives, pH adjusters, plant extracts, salts, vitamins, sunscreens, colorants, and mixtures thereof.

The silicones other than amino functional silicones can be chosen from dimethicone and dimethicone copolyol compounds such as oxypropylenated and/or oxyethylenated polydimethyl(methyl)siloxane, oxypropylenated and/or oxyethylenated polymethyl (C8-C22) alkyl dimethyl methyl siloxane, and mixtures thereof, and wherein the dimethicone copolyol compounds are preferably selected from Dimethicone PEG-8 Benzoate, Dimethicone PEG-7 Phosphate, Dimethicone PEG-8 Phosphate, Dimethicone PEG-10 Phosphate, PEG-7 Dimethicone, PEG-8 Dimethicone, PEG-9 Dimethicone, PEG-10 Dimethicone, PEG-12 Dimethicone, PEG-14 Dimethicone, PEG-17 Dimethicone, PEG/PPG-3/10 Dimethicone, PEG/PPG-4/12 Dimethicone, PEG/PPG-17/18 Dimethicone, cetyl PEG/PPG-10/1 dimethicone, and mixtures thereof.

A person skilled in the art will take care to select the optional additional components and the amount thereof such that they do not harm the properties of the compositions of the present invention.

These additional components are generally present in the composition according to the invention in an amount ranging from about 0 to about 20% by weight relative to the total weight of the composition.

In various embodiments, the hair cosmetic composition of the present invention is a conditioner composition in the form of an aqueous composition or an emulsion or a cream or a lotion.

In other various embodiments, the hair cosmetic composition of the present invention is a masque (mask) composition in the form of a thick or highly viscous emulsion or cream or lotion.

In one embodiment, the present invention is related to a method or process of conditioning hair, wherein the hair cosmetic composition is applied onto hair (wet or dry), massaged into the hair fibers, then rinsed out with water.

In one embodiment, the present invention is related to a process or method of treating or conditioning hair according to the general protocol or treatment cycle involving the steps of:

a) optionally, washing/rinsing the hair with a shampoo having a neutral pH and/or rinsing the hair with water, followed by allowing the hair to air dry, while optionally applying a smoothing action on the hair;

b) applying the composition onto the hair;

c) allowing the composition to remain on the hair for a period of time ranging from about 1 to about 10 minutes or from about 1 to about 5 minutes;

d) rinsing the hair with water;

e) drying the hair at a temperature ranging from room temperature up to about 100° C., while optionally applying a smoothing action on the hair;

f) passing a flat iron over the hair swatch at least once, or such as two times up to 10 times or such as three times up to 10 times wherein the flat iron is employed at a temperature of about 100° C., or ranging from about 100° C. to about 250° C. or from about 110° C. to about 230° C. or from about 110° C. to about 210° C. or from about 120° C. to about 200° C. or from about 150° C. to about 190° C., or from about 190° C. to about 230° C., including ranges and sub-ranges therebetween, or at a temperature of about 230° C. or about 225° C. or about 220° C. or about 210° C. or about 200° C. or about 190° C. or about 180° C. or about 150° C. or about 100° C. and preferably at about 230° C.; and

g) washing/rinsing the hair with a shampoo having a neutral pH and/or rinsing the hair with water, followed by allowing the hair to air dry, while optionally applying a smoothing action on the hair.

The smoothing action may be conducted by brushing or combing or passing the fingers through the hair.

Drying the hair at a temperature ranging from room temperature up to about 100° C. may be accomplished by drying the hair with a blow dryer device or using other heat sources such as a flat iron, a hair dryer, a heat lamp, a heat wand, or other similar devices.

In various embodiments, the flat iron is passed over the hair at least once or from one time up to 10 times or from 2 times up to 10 times or from 3 times up to 10 times or from 5 times up to 10 times or from 6 times up to 10 times or 10 times.

The composition can be applied onto the hair using an applicator device or with the hands or gloved hands or with the fingers. A suitable applicator device is an applicator brush or applicator comb or applicator spatula or a dispenser or applicator tip attached to the container holding the composition.

The method or treatment cycle described above may be repeated over a period of days or weeks.

The process of treating the hair cosmetic composition of the invention may be preceded by another cosmetic composition, such as a shampoo, which is applied onto the wet hair, left on the hair from 0 up to about 10 minutes, while optionally massaging the shampoo onto the hair fibers, then rinsed out with water.

The cosmetic effects imparted by the compositions and accompanying methods of treating the hair of the present invention may be evaluated by visually assessing the appearance of the hair after processing the hair according to the methods of the invention. Another type of evaluation can also involve sensorial evaluations of the hair.

It was surprisingly and unexpectedly discovered that the hair contacted with the compositions of the invention and treated according to the methods of the invention visually generally appeared to be more extended and/or straight, less volumized, and less frizzy compared to hair contacted with compositions that did not contain thiolactic acid. It was also surprisingly and unexpectedly discovered that the hair contacted with the compositions of the invention and treated according to the methods of the invention were smoother to the touch, more manageable, more disciplined (i.e., less or no fly-aways), and exhibited more regularity with respect to shape and appearance. These effects were even more observable after subjecting the hair to multiple treatment or application cycles.

The compositions of the present invention may be packaged in any suitable container such as a tube, a jar or a bottle. In certain embodiments, the composition can be packaged in a tube or bottle, for example, a squeeze tube or squeeze bottle. Additionally, an applicator device can be attached or connected to the opening of the packaging/squeeze tube or bottle wherein the applicator device is a brush or a comb with teeth such that the ends of the teeth have openings from which the composition of the invention can flow through and be applied directly onto the hair.

The composition of the present invention may also be provided as component of a kit for treating or cleansing/conditioning hair wherein the kit can additionally contain other components such as a shampoo.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

The compositions according to the invention can be manufactured by known processes used generally in the cosmetics, including the processes described in the examples below.

The following Examples are intended to be non-restrictive and explanatory only, with the scope of the disclosed embodiments.

The ingredient amounts in the compositions/formulas described below are expressed in % by weight, based on the total weight of the composition.

### EXAMPLES

### Example A

### Formulations

The formulas of the present invention can be prepared in water according to the table below.

TABLE 1

| | % by Weight |
|---|---|
| | (active matter, |
| Ingredient | “AM”) |
| Cationic surfactants (e.g., cetrimonium chloride, behentrimonium chloride, | 0.1-10 |
| dipalmitoylethylhydroxyethylmethylammonium methosulfate, behentrimonium | |
| methosulfate, and mixtures thereof) | |
| Thiolactic acid | 0.5-15 |
| Fatty alcohols (e.g., cetearyl alcohol, cetyl alcohol, steary alcohol, and | 6-20 |
| mixtures thereof) | |
| Polyols (eg, glycerin, propylene glycol) | 0.1-5 |
| Fatty substances other than a fatty alcohol (e.g., mineral oil, cetyl esters, and | 0.1-5 |
| mixtures thereof) | |
| Amino Silicones (eg, amodimethicone) | 0-10 |
| Cationic conditioning polymers compounds (e.g., quaternary ammonium | 0-4 |
| compounds such as polyquaternium compounds) | |
| Water, organic solvent, and additional components such as fragrance, | Q.S. 100 |
| preservatives, pH adjusting agents, plant extracts, plant oils, hydrolyzed | |
| proteins vitamins, salt, and fragrances, opacifiers (e.g., glycol distearate), | |
| plasticizers, salts, vitamins, sunscreens, lower alkanes, hydrocarbons, | |
| colorants, or mixtures thereof (as needed or desired) | |

Examples of the inventive rinse-off conditioner formulas containing 1, 2.5, 5, and 8% by weight of thiolactic acid, based on the total weight of the composition, and comparative formulas which do not contain thiolactic acid were prepared and are presented in the table below.

TABLE 2
Rinse-off Conditioner Formulas

| | Comparative | | | | |
|---|---|---|---|---|---|
| | formula | Inventive formulas | | | |
| | Example 1 | Example 2 | Example 3 | Example 4 | Example 5 |
| US INCI NAME | % by weight | | | | |
| THIOLACTIC ACID | — | 1 | 2.5 | 5 | 8 |
| BEHENTRIMONIUM CHLORIDE | 2.37 | 2.37 | 2.37 | 2.37 | 2.37 |
| CETEARYL ALCOHOL | 7 | 7 | 7 | 7 | 7 |
| GLYCERIN | 0.5 | 0.5 | 0.5 | 0.5 | 0.5 |
| SYNTHETIC FLUORPHLOGOPITE | 3 | 3 | 3 | 3 | 3 |
| (and) TITANIUM DIOXIDE | | | | | |
| WATER AND ADDITIONAL | QS 100 | QS 100 | QS 100 | QS 100 | QS 100 |
| COMPONENT (PRESERVATIVES) | | | | | |
| pH | 3.5 | 3.5 | 3.5 | 3.5 | 3.5 |

TABLE 3
Rinse-off Conditioner Formulas with Amino silicone

| | Comparative | Inventive formulas | | | |
|---|---|---|---|---|---|
| | formula | | | | Example |
| | Example 6 | Example 7 | Example 8 | Example 9 | 10 |
| US INCI NAME | % by weight | | | | |
| THIOLACTIC ACID | — | 1 | 2.5 | 5 | 8 |
| BEHENTRIMONIUM CHLORIDE | 2.37 | 2.37 | 2.37 | 2.37 | 2.37 |
| CETEARYL ALCOHOL | 7 | 7 | 7 | 7 | 7 |
| AMODIMETHICONE (and) | 2 | 2 | 2 | 2 | 2 |
| TRIDECETH-6 (and) | | | | | |
| CETRIMONIUM CHLORIDE | | | | | |
| (XIAMETER ® MEM-8299 Cationic | | | | | |
| Emulsion from Dow Corning; 57.5% | | | | | |
| AM) | | | | | |
| GLYCERIN | 0.5 | 0.5 | 0.5 | 0.5 | 0.5 |
| SYNTHETIC FLUORPHLOGOPITE | 3 | 3 | 3 | 3 | 3 |
| (and) TITANIUM DIOXIDE | | | | | |
| WATER AND ADDITIONAL | QS 100 | QS 100 | QS 100 | QS 100 | QS 100 |
| COMPONENTS (ISOPROPYL | | | | | |
| ALCOHOL, PRESERVATIVES) | | | | | |
| pH | 3.5 | 3.5 | 3.5 | 3.5 | 3.5 |

TABLE 4
Rinse-off Conditioner Mask Formulas

| | Comparative | Inventive formulas | | | |
|---|---|---|---|---|---|
| | formula | | | | Example |
| | Example 11 | Example 12 | Example 13 | Example 14 | 15 |
| US INCI NAME | % by weight | | | | |
| THIOLACTIC ACID | — | 1 | 2.5 | 5 | 8 |
| CETTRIMONIUM CHLORIDE | 0.8 | 0.8 | 0.8 | 0.8 | 0.8 |
| DIPALMITOYLETHYL | 1.35 | 1.35 | 1.35 | 1.35 | 1.35 |
| HYDROXYETHYLMONIUM | | | | | |
| METHOSULFATE* | | | | | |
| CETEARYL ALCOHOL | 9.15 | 9.15 | 9.15 | 9.15 | 9.15 |
| CETYL ESTERS (and) CETYL | 1 | 1 | 1 | 1 | 1 |
| ESTERS | | | | | |
| MINERAL OIL | 2 | 2 | 2 | 2 | 2 |
| WATER AND ADDITIONAL | QS 100 | QS 100 | QS 100 | QS 100 | QS 100 |
| COMPONENT (PRESERVATIVES) | | | | | |
| pH | 3.5 | 3.5 | 3.5 | 3.5 | 3.5 |

*available under the tradename DEHYQUART F 30 and commercially available from BASF or under the tradename TETRANYL L6-30 and commercially available from Kao

The formulas in the tables 2 and 3 were made according to the procedure:

The cationic surfactant (behentrimonium and/or ceterimonium chloride) was added to water to form an aqueous solution. When an amino silicone such as amodimethicone (in emulsion form) was present, the amino silicone was stirred into the solution for a few minutes. As a separate mixture, cetearyl alcohol was added to water (in an amount of about 50% by weight of the total amount of water in the formula) with fast stirring for about 15 minutes at 70° C. Glycerin was added and mixed to thicken the mixture. Finally, thiolactic acid was added at room temperature. If necessary, pH was adjusted with Sodium Hydroxide.

The formulas in the table 4 were made according to the procedure:

Cetearyl alcohol, cetyl esters, mineral oil and dipalmitoylethylhydroxyethylmonium methosulfate were mixed with water (20% by wt of the total amount of water in the formula) with heating at 70° C. to form an aqueous solution. Cetrimonium chloride was added with fast stirring for 15 minutes. 80% by wt of the total amount of water in the formula was added to cool the system to room temperature. Finally, thiolactic acid was added at room temperature. If necessary, pH was adjusted with Sodium Hydroxide.

Optional ingredients and additives were included in the formulas: for example, organic solvents, fragrance, preservatives, plant oils and extracts, neutralizing agents, cationic conditioning polymers, silicones, opacifiers, plasticizers, salts, vitamins, sunscreens, lower alkanes, hydrocarbons, colorants, or mixtures thereof.

### Example B

### Performance Evaluation

Application Protocol

The formulas above were used to treat hair swatches according to the following hair treatment protocol:

1. The hair swatches were washed with a shampoo having a neutral pH, rinsed with water, and the hair was dried at a temperature ranging from room temperature up to about 100° C. by using for example, a blow dryer, while optionally applying a smoothing action on the hair.

2. Each formula was spread along the length of designated wet hair swatches and allowed to remain on the hair for 5 minutes.

3. The hair swatches were rinsed with water and the hair was dried at a temperature ranging from room temperature up to about 100° C. by using for example, a blow dryer, while optionally applying a smoothing action on the hair.

4. A flat iron was passed over each hair swatch 3 times at 230° C.

5. The hair swatches were washed with a shampoo having a neutral pH, rinsed with water, and allowed to air dry.

6. The hair swatches were subjected to sensorial and visual assessments with respect to the attributes of straightening, volume (i.e., volume control or reduction) and frizz (i.e., frizz control or reduction) based on a standardized rating scale of 1 to 4, with 4 being the best rating (highest degree of straightening, least amount of volume and least amount of frizziness).

In order to evaluate the long-lasting performance of the formulas on hair over time, the hair swatches were also subjected to multiple applications (from 2 to 5 applications) according to the protocol above based on the common practice of people of washing/cleansing their hair 2 to 3 times a week. One application corresponds to one treatment cycle comprising steps 1 to 5. Five applications or five treatment cycles were designed to simulate washing/cleansing of hair over a two week period (people tend to wash their hair 2 to 3 times a week).

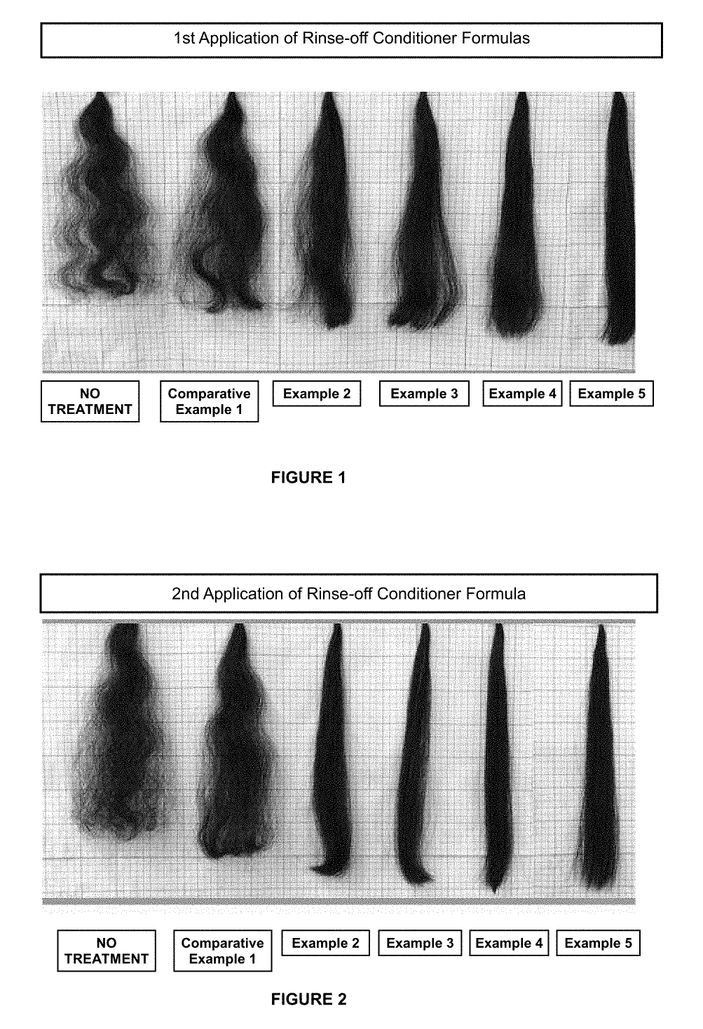

FIGS. 1 to 3 are photographic images of the hair swatches treated with the inventive and comparative conditioner formulas in Table 2 (Examples 1 to 5) and subjected to several applications or treatment cycles (at 1st, 2nd, and 5th applications).

FIG. 4 presents photographic images of the hair swatches treated with the inventive and comparative conditioner formulas in Table 3 that contain amino silicone (Examples 6 to 10).

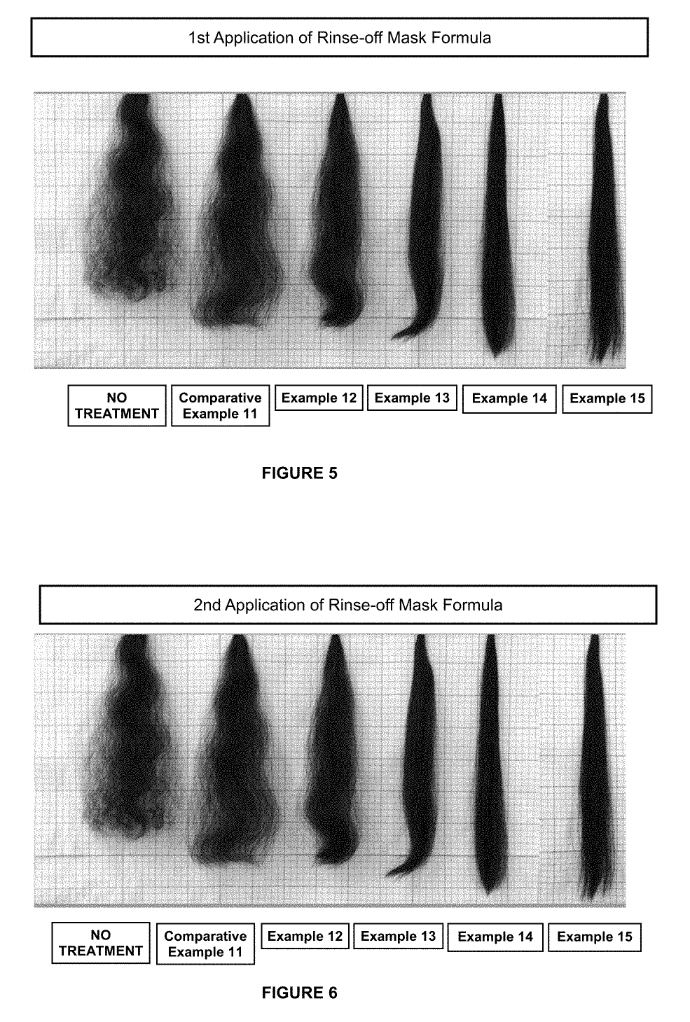

FIGS. 5 to 7 are photographic images of the hair swatches treated with the inventive and comparative conditioner formulas in Table 3 (Examples 11 to 15) and subjected to several applications or treatment cycles (at 1st, 2nd, and 5th applications).

Performance Results

I. Rinse-Off Conditioner Formulas

A. First Application

| | No | Compar- | | | | |
|---|---|---|---|---|---|---|
| | treatment | ative | | | | |
| | (water | Exam- | Exam- | Exam- | Exam- | Exam- |
| Attributes | only) | ple 1 | ple 2 | ple 3 | ple 4 | ple 5 |
| Straight | 1.0 | 1.5 | 1.5 | 2.5 | 3.0 | 3.5 |
| Volume | 1.0 | 1.5 | 2.5 | 3.5 | 4.0 | 3.5 |
| Frizz | 1.0 | 2.0 | 3.0 | 3.0 | 3.5 | 3.0 |

Sensorial & Visual Evaluations

Straightening

Manageability

Volume Reduction

Frizz Control

Smooth Touch

High Cosmeticity

Regularity

Discipline

B. Second Application

| | No | Compar- | | | | |
|---|---|---|---|---|---|---|
| | treatment | ative | | | | |
| | (water | Exam- | Exam- | Exam- | Exam- | Exam- |
| Attributes | only) | ple 1 | ple 2 | ple 3 | ple 4 | ple 5 |
| Straight- | 1.0 | 1.5 | 3.5 | 3.5 | 4.0 | 4.0 |
| ening | | | | | | |
| Volume | 1.0 | 1.5 | 3.5 | 4.0 | 4.0 | 3.5 |
| Frizz | 1.0 | 3.0 | 3.5 | 3.5 | 4.0 | 3.5 |

Sensorial & Visual Evaluations

Straightening

Volume Reduction

Frizz Control

Smooth Touch

High Cosmeticity

Regularity

Discipline

Natural Touch

C. Fifth Application

| | No | Compar- | | | | |
|---|---|---|---|---|---|---|
| | treatment | ative | | | | |
| | (water | Exam- | Exam- | Exam- | Exam- | Exam- |
| Attributes | only) | ple 1 | ple 2 | ple 3 | ple 4 | ple 5 |
| Straight- | 1.0 | 3.0 | 3.0 | 3.0 | 4.0 | 4.0 |
| ening | | | | | | |
| Volume | 1.0 | 3.0 | 3.5 | 3.0 | 3.5 | 4.0 |
| Frizz | 1.0 | 3.0 | 3.5 | 3.5 | 3.0 | 3.5 |

Sensorial & Visual Evaluations

Straightening

Volume Reduction

Frizz Control

Smooth Touch

High Cosmeticity

Regularity

Discipline

Natural Touch

Summary of Results:

After the 1st and 2^{nd} applications, the ratings for the hair swatches treated with the inventive formulas at 1, 2.5, 5, and 8% by weight of thiolactic acid were much higher than the ratings for the hair treated with the comparative formulas with respect to volume and frizz. Also, the ratings for the hair swatches treated with the inventive formulas at 2.5, 5, and 8% by weight of thiolactic acid were much higher than the ratings for the hair treated with the comparative formulas with respect to straightening. The rating on the straightening attribute for the hair swatch treated with 1% by weight of thiolactic acid was comparable to the rating for the swatch treated with the comparative example. These effects are visually evident in FIGS. 1 and 2.

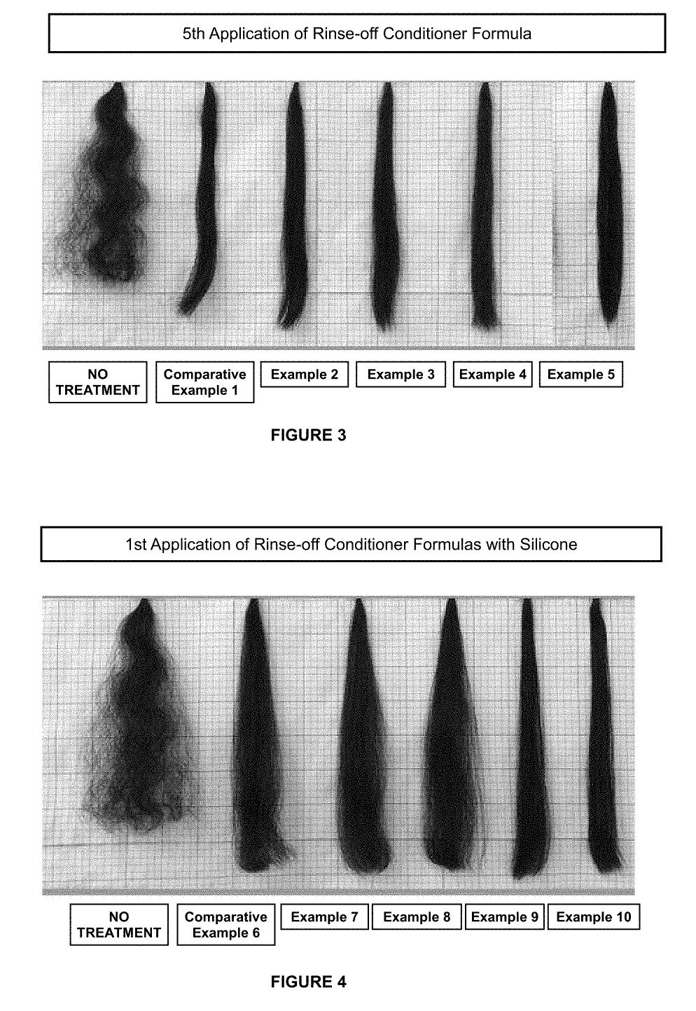

After the 5th application, the ratings for the hair swatches treated with the inventive formulas at 1, 2.5, 5, and 8% by weight of thiolactic acid were comparable or higher than the ratings for the hair treated with the comparative formula with respect to straightening, volume and frizz. These effects are visually evident in FIG. 3.

The effects of multiple applications were also observed, especially after the 2nd application wherein the ratings and the visual appearance of the hair swatches treated with the inventive formulas at 1, 2.5, 5, and 8% by weight of thiolactic acid significantly improved from the 1st application for most of the attributes of straightening, volume and frizz. At the fifth application, the ratings were generally comparable to the ratings after the 2nd application. These effects are visually evident in FIGS. 1 to 3. While the rest of the results showed lower ratings for the hair treated with the inventive shampoos with respect to some of the attributes at various application cycles and at certain levels of thiolactic acid,

While some of the visual ratings on straightening, volume and frizz for hair treated with the inventive examples were comparable or the same as those for the hair treated with the comparative example, the hair treated with the inventive formulas provided better sensorial (by feel) and other visual attributes of high cosmeticity, smooth touch, natural touch, regularity and discipline as compared to the hair treated with the comparative formulas that did not contain thiolactic acid.

Overall, the conditioner formulas of the invention imparted hair care and manageability properties to the hair by providing the cosmetic effects of straightening, volume control or reduction, frizz control or less frizziness, high cosmeticity, smooth touch, discipline, regularity and natural touch.

II. Rinse-off Conditioner Formulas with Silicone

A. First Application

| | No | Compar- | | | | |
|---|---|---|---|---|---|---|
| | treatment | ative | | | | |
| | (water | Exam- | Exam- | Exam- | Exam- | Exam- |
| Attributes | only) | ple 6 | ple 7 | ple 8 | ple 9 | ple 10 |
| Straight | 1.0 | 3.0 | 2.5 | 2.5 | 3.5 | 3.5 |
| Volume | 1.0 | 2.0 | 2.0 | 2.0 | 4.0 | 4.0 |
| Frizz | 1.0 | 2.5 | 2.5 | 3.0 | 3.5 | 3.5 |

Sensorial & Visual Evaluations

Straightening

High Volume Reduction

Frizz Control

High Smooth Touch

High Cosmeticity

Regularity

Discipline

Summary of Results:

After the 1st application, the ratings for the hair swatches treated with the inventive formulas at 5 and 8% by weight of thiolactic acid were much higher than the ratings for the hair treated with the comparative formulas with respect to straightening, volume and frizz. For the hair treated with formulas having 1 and 2.5% by weight of thiolactic acid, the ratings for these attributes were mostly the same or comparable to the ratings for the hair treated with the comparative example. These effects are visually evident in FIG. 4.

Nevertheless, while some of the visual ratings on straightening, volume and frizz for hair treated with the inventive examples were comparable or the same as those for the hair treated with the comparative example, the hair treated with the inventive formulas provided better sensorial (by feel) and other visual attributes of high cosmeticity, high smooth touch, regularity and discipline as compared to the hair treated with the comparative formulas that did not contain thiolactic acid.

Overall, the conditioner formulas of the invention which contained thiolactic acid imparted hair care and manageability properties to the hair by providing the cosmetic effects of straightening, volume control or reduction, frizz control or less frizziness, high cosmeticity, high smooth touch, discipline, and regularity

III. Rinse-off Mask Formulas

A. First Application

| | No | Compar- | | | | |
|---|---|---|---|---|---|---|
| | treatment | ative | | | | |
| | (water | Exam- | Exam- | Exam- | Exam- | Exam- |
| Attributes | only) | ple 11 | ple 12 | ple 13 | ple 14 | ple 15 |
| Straight | 1.0 | 1.0 | 2.0 | 2.5 | 2.5 | 3.5 |
| Volume | 1.0 | 3.0 | 3.0 | 3.0 | 3.0 | 4.0 |
| Frizz | 1.0 | 2.5 | 2.5 | 3.0 | 3.0 | 3.5 |

Sensorial & Visual Evaluations

Straightening

Manageability

Volume Reduction

Frizz Control

Very Smooth Touch

High Cosmeticity

Regularity

Discipline

B. Second Application

| | No | Compar- | | | | |
|---|---|---|---|---|---|---|
| | treatment | ative | | | | |
| | (water | Exam- | Exam- | Exam- | Exam- | Exam- |
| Attributes | only) | ple 11 | ple 12 | ple 13 | ple 14 | ple 15 |
| Straight- | 1.0 | 1.5 | 2.0 | 3.0 | 4.0 | 3.5 |
| ening | | | | | | |
| Volume | 1.0 | 2.0 | 2.0 | 3.5 | 4.0 | 4.0 |
| Frizz | 1.0 | 2.0 | 2.5 | 3.5 | 4.0 | 3.5 |

Sensorial & Visual Evaluations

Straightening

Manageability

Volume Reduction

Frizz Control

Very Smooth Touch

High Cosmeticity

Regularity

Discipline

C. Fifth Application

| | No | Compar- | | | | |
|---|---|---|---|---|---|---|
| | treatment | ative | | | | |
| | (water | Exam- | Exam- | Exam- | Exam- | Exam- |
| Attributes | only) | ple 11 | ple 12 | ple 13 | ple 14 | ple 15 |
| Straight- | 1.0 | 3.5 | 3.5 | 3.5 | 3.5 | 3.5 |
| ening | | | | | | |
| Volume | 1.0 | 3.5 | 3.5 | 4.0 | 4.0 | 3.5 |
| Frizz | 1.0 | 3.0 | 3.5 | 3.5 | 3.0 | 3.5 |

Sensorial & Visual Evaluations

Straightening

Volume Reduction

Frizz Control

Very Smooth Touch

High Cosmeticity

Regularity

Discipline

Summary of Results:

After the 1st application, the ratings for the hair swatches treated with the inventive formulas at 1, 2.5, 5, and 8% by weight of thiolactic acid were comparable or higher than the ratings for the hair treated with the comparative formulas with respect to straightening, volume and frizz. After the 2^{nd} application, the ratings for the hair swatches treated with the inventive formulas at 5, 5, and 8% by weight of thiolactic acid were much higher than the ratings for the hair treated with the comparative formulas with respect to straightening, volume and frizz. At 1% by weight of thiolactic acid, the ratings for the hair treated with the inventive formulas were comparable or higher than the ratings for hair treated with the comparative. These effects are visually evident in FIGS. 5 and 6.

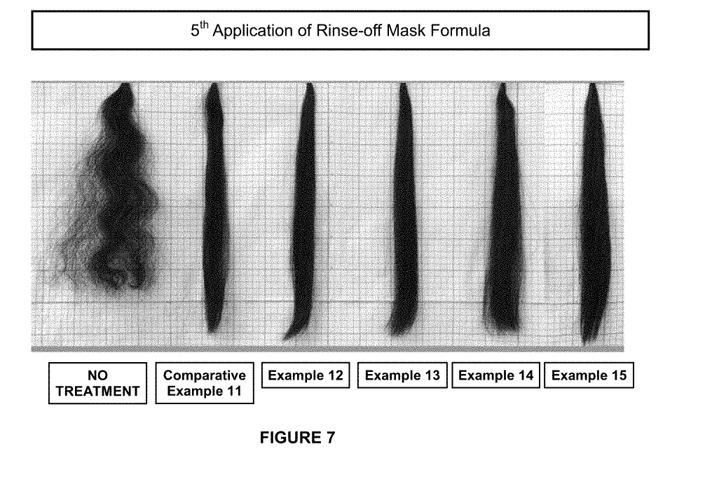

After the 5th application, the ratings for the hair swatches treated with the inventive formulas at 1, 2.5, 5, and 8% by weight of thiolactic acid were comparable or higher than the ratings for the hair treated with the comparative formula with respect to straightening, volume and frizz. These effects are visually evident in FIG. 7.

The effects of multiple applications were also observed: with increasing number of applications (from 1^{st} to 5^{th} application), the ratings and the visual appearance of the hair swatches treated with the inventive formulas at 2.5, 5, and 8% by weight of thiolactic acid improved for the attributes of straightening, volume and frizz. The improvement was more significant in the ratings at 1^{st} application compared to the 5^{th} application at 1, 2.5, 5, and 8% by weight of thiolactic acid for all three attributes. These effects are visually evident in FIGS. 5 to 7. While similar multi-application effects were also observed from the treatment with the comparative example, the hair treated with the inventive formulas provided better sensorial (by feel) and other visual attributes of high cosmeticity, very smooth touch, regularity and discipline as compared to the hair treated with the comparative formulas that did not contain thiolactic acid.

Overall, the conditioner formulas of the invention imparted hair care and manageability properties to the hair by providing the cosmetic effects of straightening, volume control or reduction, frizz control or less frizziness, high cosmeticity, very smooth touch, discipline, and regularity.

1. A hair cosmetic composition comprising:
(a) at least one cationic surfactant;
(b) at least one thiol-based compound selected from thiolactic acid, thiolactic acid derivatives, their salts, and mixtures thereof;
(c) at least one fatty alcohol in an amount of from about 6% to about 20% by weight;
(d) at least component selected from (i) polyols, (ii) fatty substances other than a fatty alcohol, and mixtures thereof in an amount of from about 0.1% to about 10% by weight;
(e) water; and
(f) optionally, at least one neutralizing agent;
wherein the pH of the composition ranges from 2 to less than 7; all weights being relative to the total weight of the composition.

2. The hair cosmetic composition according to claim 1, wherein the at least one cationic surfactant is selected from optionally polyoxyalkenylated primary, secondary, tertiary fatty amine salts, quaternary ammonium compounds and their salts, and mixtures thereof.

3. The hair cosmetic composition according to claim 1, wherein the at least one cationic surfactant is present in an amount of from about 0.1% to about 10% by weight, relative to the total weight of the composition.

4. The hair cosmetic composition according to claim 1, wherein the at least one thiol-based compound is selected from thiolactic acid.

5. The hair cosmetic composition according to claim 1, wherein the at least one thiol-based compound is present in an amount of from about 0.5% to about 15% by weight, based on the total weight of the composition.

6. The hair cosmetic composition according to claim 1, wherein the at least one fatty alcohol is selected from linear saturated/unsaturated fatty alcohols comprising from 6 to 60 carbon atoms.

7. The hair cosmetic composition according to claim 1, wherein the at least one fatty alcohol is present in an amount of from about 6.5% to about 15% by weight, relative to the total weight of the composition.

8. The hair cosmetic composition according to claim 1, where the at least one component (d) comprises:
(i) polyols selected from ethylene glycol, propylene glycol, 1,3-butylene glycol, pentylene glycol, hexylene glycol, dipropylene glycol, glycerin, and mixtures thereof; and/or
(ii) fatty substances other than a fatty alcohol and selected from hydrocarbons, fatty esters, plant/vegetable oils, esters of fatty acid, alkanes, waxes, non-silicone synthetic oils, and mixtures thereof;
(iii) and mixtures thereof.

9. The hair cosmetic composition according to claim 1, wherein the at least one component (d) is present in an amount of from about 0.1% to about 10% by weight, relative to the total weight of the composition.

10. The composition, according to claim 1, further comprising at least one neutralizing agent selected from organic amines, alkali metal hydroxides, alkali earth metal hydroxides, alkali metal carbonates, alkali metal phosphates, and mixtures thereof.

11. (canceled)

12. The hair cosmetic composition according to claim 1, further comprising at least one amino silicone selected from amodimethicone, trideceth-9 PG amodimethicone, PEG-40/PPG-8 Methylaminopropyl/hydroxypropyl dimethicone copolymer, and mixtures thereof.

13. (canceled)

14. The hair cosmetic composition according to claim 1, further comprising at least one cationic conditioning polymer selected from cationic cellulose derivatives, cationic gum derivatives, polymer derivatives of diallyldimethyl ammonium chloride, polymer derivatives of methacrylamidopropyltrimethylammonium chloride, and mixtures thereof.

15. (canceled)

16. The composition, according to claim 1, wherein the pH of the composition ranges from about pH 2.5 to about 6.5.

17. A hair cosmetic composition comprising:
(a) at least one cationic surfactant selected from quaternary ammonium compounds and their salts present in an amount of from about 0.5% to about 8% by weight;
(b) at least one thiol-based compound selected from thiolactic acid, thiolactic acid derivatives, their salts, and mixtures thereof and present in an amount of from about 0.7% to about 12% by weight;
(c) at least one fatty alcohol selected from linear saturated/unsaturated fatty alcohols comprising from 6 to 60 carbon atoms in an amount of from about 6.5% to about 15% by weight;
(d) at least component selected from (i) polyols; (ii) fatty substances other than a fatty alcohol; and (iii) and mixtures thereof in an amount of from about 0.2% to about 8% by weight;
(e) water; and
(f) optionally, at least one neutralizing agent;
wherein the pH of the composition ranges from about 3 to about 5;
all weights being relative to the total weight of the composition.

18. The hair cosmetic composition according to claim 17, wherein the composition further comprises at least one amino silicone selected from amodimethicone in an amount of from about 0.05% to about 3% by weight, relative to the total weight of the composition.

19. The hair cosmetic composition according to claim 17, wherein the at least component (d) comprises polyols selected from ethylene glycol, propylene glycol, 1,3-butylene glycol, pentylene glycol, hexylene glycol, dipropylene glycol, glycerin, and mixtures thereof.

20. (canceled)

21. The hair cosmetic composition according to claim 1, wherein the composition is substantially free of anionic surfactants selected from sulfate surfactants, sulfonate surfactants, sarcosinate surfactants, and mixtures thereof.

22. (canceled)

23. A method of treating hair, the method comprising a treatment cycle involving the steps of:
a) optionally, washing/rinsing the hair with a shampoo having a neutral pH and/or rinsing the hair with water, followed by allowing the hair to air dry, while optionally applying a smoothing action on the hair;
b) applying the composition of claim 1 onto the hair;
c) allowing the composition in b) to remain on the hair for a period of time ranging from 1 to 10 minutes or from 1 to 5 minutes;
d) rinsing the hair with water;
e) drying the hair at a temperature ranging from room temperature up to about 100° C., while optionally applying a smoothing action on the hair;
f) passing a flat iron over the hair swatch at least three times; and
g) optionally, washing/rinsing the hair with a shampoo having a neutral pH and/or rinsing the hair with water, followed by allowing the hair to air dry, while optionally applying a smoothing action on the hair.

24. The method of the preceding claim, wherein the flat iron is employed at a temperature ranging from 100° C. to 250° C.

25. (canceled)

26. The method according to claim 23, wherein the treatment cycle is repeated over a period of days.

27. (canceled)

28. (canceled)
